(19) United States
(12) Patent Application Publication
(10) Pub. No.: US 2003/0068446 A1
(43) Pub. Date: Apr. 10, 2003

(54) Protein and peptide nanoarrays

(75) Inventors: Chad A. Mirkin, Evanston, IL (US); Guy Della Cioppa, Chicago, IL (US); Linette Demers, Chicago, IL (US); Ki-Bum Lee, Evanston, IL (US); So-Jung Park, Evanston, IL (US)

Correspondence Address: FOLEY AND LARDNER, SUITE 500, 3000 K STREET NW, WASHINGTON, DC, 20007, US

(73) Assignee: Northwestern University

(21) Appl. No.: 10/261,663

(22) Filed: Oct. 2, 2002

Related U.S. Application Data
(60) Provisional application No. 60/326,767, filed on Oct. 2, 2001.

(51) Int. Cl. B05D 1/18; A61L 2/00

(52) U.S. Cl. 427/430.1; 427/2.1

(57) ABSTRACT

Ultrahigh resolution patterning, preferably carried out by dip-pen nanolithographic printing, can be used to construct peptide and protein nanoarrays with nanometer-level dimensions. The peptide and protein nanoarrays, for example, exhibit almost no detectable nonspecific binding of proteins to their passivated portions. This work demonstrates how dip pen nanolithographic printing can be used in a method to generate high density protein and peptide patterns, which exhibit bioactivity and virtually no non-specific adsorption. It also shows that one can use AFM-based screening procedures to study the reactivity of the features that comprise such nanoarrays. The method encompasses a wide range of protein and peptide structures including, for example, enzymes and antibodies. Features at or below 300 nm can be achieved.

This application claims benefit of provisional application 60/326,767, filed Oct. 2, 2001 and U.S. application Ser. No. 09/866,533, filed May 24, 2001, the complete disclosures of which are incorporated herein by reference in their entireties.

### FIELD OF THE INVENTION

This invention relates to nanoarrays of proteins and peptides, methods of making them, and uses thereof. The invention also relates to DIP PEN™ nanolithographic printing (DPN™ and DIP PEN NANOLITHOGRAPHY™ are trademarks of Nanolnk, Inc.; Chicago, Ill.).

### BACKGROUND

The development of DIP PEN™ nanolithographic printing and the preparation of arrays are described in priority application Ser. No. 09/866,533, filed May 24, 2001, particularly in the “Background of the Invention” section (pages 1-3), which is hereby incorporated by reference in its entirety.

In addition, the development of protein and peptide arrays, microarrays, and nanoarrays is described, with literature citations, in priority application 60/326,767, filed Oct. 2, 2001, including the use of DIP PEN™ nanolithographic printing to generate protein and peptide nanoarrays, which is hereby incorporated by reference in its entirety.

Protein and peptide arrays and microarrays are important to the biotechnology and pharmaceutical industries and find applications in, for example, proteomics, pharmaceutical screening processes, diagnostics, therapeutics, and panel immunoassays. Nanoarrays, however, are less well developed, and the production of protein and peptide nanoarrays is an important commercial goal of nanotechnology.

A variety of patterning techniques have been used in attempts to fabricate such arrays including photolithography, microcontact printing, nanografting, and spot arraying. However, attempted miniaturization in making protein and peptide nanoarrays can generate significant problems. Technology suitable for large scale array manufacture may not be suitable for nanoarray manufacture. For example, miniaturization can increase nonspecific binding to the array, distorting experimental and diagnostic results. Nonspecific background noise can make it difficult to differentiate inactive areas of the array, thereby complicating analysis of nanoscale libraries. Also, soft materials used in some of these technologies may not allow for nanoscale production. Finally, traditional optical screening methods may not work.

Despite the difficulties, protein and peptide nanoarrays having features less than, for example, 1,000 nm, and preferably less than 300 nm, represent a commercially important target. They would increase peptide and protein library density and expand library analysis. The methods used to prepare these structures should be generally free from the problems associated with conventional nanotechnology such as, for example, electron beam lithography.

### SUMMARY

The present invention provides for nanoscopic peptide and protein nanoarrays which, preferably, are prepared with use of DIP PEN™ nanolithographic printing. One advantage of the inventions herein is the wide variety of different embodiments, reflecting the versatility of the DIP PEN™ nanolithographic printing method and the wide spectrum of peptide chemistry. The nanoarrays comprise high density peptide and protein patterns, which exhibit bioactivity and virtually no non-specific adsorption.

For example, a protein nanoarray is provided comprising: (a) a nanoarray substrate, (b) a plurality of dots on the substrate, the dots comprising at least one patterning compound on the substrate, and at least one protein on the patterning compound. The patterning compound can be placed on the substrate by DIP PEN™ nanolithographic printing, and the plurality of dots can be in the form of a lattice.

The present invention also provides a protein nanoarray comprising: (a) a nanoarray substrate, (b) a plurality of lines on the substrate, the lines comprising at least one patterning compound on the substrate and at least one protein on the patterning compound. The patterning compound can be placed on the substrate by DIP PEN™ nanolithographic printing, and the plurality of lines can be in the form of a grid with perpendicular or parallel lines.

More generally, the protein nanoarrays comprise a nanoarray substrate, and a plurality of patterns on the substrate, and the patterns comprise at least one patterning compound on the substrate and at least one protein adsorbed to each of the patterns.

More generally, peptide nanoarrays are also provided. For example, the invention provides a peptide nanoarray comprising: a) a nanoarray substrate, b) a plurality of dots on the substrate, the dots comprising at least one compound on the substrate, and at least one peptide adsorbed to each of the dots.

Also, a peptide nanoarray is provided comprising: a) a nanoarray substrate, b) a plurality of lines on the substrate, the lines comprising at least one compound on the substrate and at least one peptide on the compound.

In another embodiment, a peptide nanoarray is provided comprising: a nanoarray substrate, at least one pattern on the substrate, the pattern comprising a patterning compound covalently bound to or chemisorbed to the substrate, the pattern comprising a peptide adsorbed on the patterning compound.

The peptide can be, for example, protein, polypeptide, or oligopeptide. Peptides can be compounds that have, for example, 100-300 peptide bonds.

The present invention also provides a method for making a nanoarray comprising: (a) patterning a compound on a nanoarray surface by DIP PEN™ nanolithographic printing to form a pattern; and (b) assembling at least one peptide onto the pattern (i.e., “method 1”).

The present invention also provides a method comprising: (a) patterning a compound on a nanoarray surface using a coated atomic force microscope tip to form a plurality of nanoscale patterns, and (b) adsorbing one or more peptides onto the pattern (i.e., “method 2”).

The present invention also provides a method for making protein nanoarrays with nanoscopic features comprising assembling one or more proteins onto a preformed nanoarray pattern, wherein the protein becomes adsorbed to the pattern and the pattern is formed by DIP PEN™ nanolithographic printing (i.e., “method 3”).

Still further, the present invention also provides a method for making peptide arrays with nanoscopic features comprising assembling one or more peptides onto a preformed nanoarray pattern, wherein the peptide becomes adsorbed to the pattern and the pattern is formed by DIP PEN™ nanolithographic printing (i.e., “method 4”).

The present invention also provides a method for making a nanoscale array of protein comprising: (a) depositing by dip-pen nanolithographic printing a patterning compound on a nanoarray surface; (b) passivating the undeposited regions of the surface with a passivation compound; (c) exposing said surface having the patterning compound and the passivation compound to a solution comprising at least one protein; (d) removing said surface from said solution of protein, wherein said surface comprises a nanoscale array of protein (i.e., “method 5”).

The present invention also provides for articles, arrays, and nanoarrays prepared by method 1, by method 2, by method 3, method 4, and by method 5.

Also provided is a submicrometer array comprising: a plurality of discrete sample areas arranged in a pattern on a substrate, each sample area being a predetermined shape, at least one dimension of each of the sample areas, other than depth, being less than about one micron, wherein each of the sample areas comprise a patterning compound on the substrate and a peptide on the patterning compound.

Furthermore, a peptide nanoarray is provided comprising:

a) a nanoarray substrate, b) a plurality of patterns on the substrate, the patterns comprising at least one patterning compound on the substrate having a terminal functional group and at least one peptide bound to each of the patterns through the terminal functional group.

Nanoscale arrays of proteins and nanoarrays find a variety of uses, including detecting whether or not a target is in a sample. For example, the present invention also provides a method for detecting the presence or absence of a target in a sample, comprising: (a) exposing a nanoarray substrate surface to a sample, the substrate surface comprising a plurality of one or more peptides assembled on one or more compounds anchored to said substrate surface, (b) observing whether a change in a property occurs upon the exposure which indicates the presence or absence of the target in the sample.

In addition, also provided is a method for detecting the presence or absence of a target in a sample, comprising: (a) exposing a nanoarray substrate surface to (i) the sample which may or may not comprise the target, and (ii) a molecule that is capable of interacting with the target, wherein the substrate surface comprises one or more peptides assembled on one or more compounds anchored to said substrate surface and the peptides are capable of binding to the target, (b) detecting the presence or absence of the target in the sample based on interaction of the molecule with the target, the target being bound to the peptide.

Finally, a method is provided for detecting the presence or absence of a target in a sample, comprising: (a) measuring at least one dimension of one or more nanoscale deposits of peptides on a surface; (b) exposing said surface to said sample; and (c) detecting whether a change occurs in the dimension of the one or more nanoscale deposits of peptides which indicates the presence or absence of the target.

Basic and novel features of the invention, particularly when DIP PEN™ nanolithographic printing is used, are many. For example, DIP PEN™ nanolithographic printing can deliver relatively small amounts of a molecular substance to a substrate in a nanolithographic fashion, at high resolution, without relying on a resist, a stamp, complicated processing methods, or sophisticated non-commercial instrumentation. In many embodiments, the invention also consists essentially of the elimination of these and other steps so prevalent in the prior art and competitive technologies. Nanometer technology is enabled, including dimensions down to and below 100 nm, as opposed to mere micron level technology.

Still further, the invention shows that AFM-based screening procedures can be used to study the reactivity of features that comprise the nanoarrays.

Finally, the invention can be carried out with a wide variety of peptide and protein structures including many antibodies which have been used in conventional histochemical assays.

### BRIEF DESCRIPTION OF THE DRAWINGS

FIG. 1: An illustration of the use of DIP PEN™ nanolithographic printing to generate structures used for subsequent passivation and peptide and protein adsorption steps to make peptide and protein nanoarrays.

FIG. 2: AFM images and height profiles of Lysozyme nanoarrays.

(A) Lateral force image of a 8 μm by 8 μm square lattice of MHA dots deposited onto an Au substrate. The array was imaged with a bare tip at 42% relative humidity (scan rate=4 Hz).

(B) Topography image (contact mode) and height profile of the nanoarray after Lysozyme adsorption. A tip-substrate contact force of 0.2 nN was used to avoid damaging the protein patterns with the tip.

(C) A tapping mode image (silicon cantilever, spring constant=˜40 N/m) and height profile of a hexagonal Lysozyme nano array. The image was taken at 0.5 Hz scan rate to obtain high resolution.

(D) Three-dimensional topographic image of a Lysozyme nanoarray, consisting of a line grid and dots with intentionally varied feature dimensions. Imaging was done in contact mode as described in (B).

FIG. 3: (A) AFM tapping mode image and height profile of IgG assembled onto an MHA dot array generated. The scan speed was 0.5 Hz.

(B) Three-dimensional topographic image of the same area displayed in (A).

(C) AFM tapping mode image and height profile of anti-IgG attached biospecifically onto the IgG nanoarray, displayed in (A) and (B). The height profile shows that the height after reaction is 16±0.9 nm (n=10). Writing and imaging conditions were the same as in (A).

(D) Three-dimensional topographic image for the area displayed in C.

FIG. 4 shows a tapping mode image and height profile of a hexagonal Lysozyme nanoarray.

FIG. 5 shows (A) a topography image (contact mode) of a IgG nanoarray, (B) three-dimensional topographic image of the same area displayed in 32(A).

### DETAILED DESCRIPTION

In priority application Ser. No. 09/866,533, filed May 24, 2001, DIP PEN™ nanolithographic printing background and procedures are described in detail covering a wide variety of embodiments including, for example:

background (pages 1-3);

summary (pages 3-4);

brief description of drawings (pages 4-10);

use of scanning probe microscope tips (pages 10-12);

substrates (pages 12-13);

patterning compounds (pages 13-17);

practicing methods including, for example, coating tips (pages 18-20);

instrumentation including nanoplotters (pages 20-24);

use of multiple layers and related printing and lithographic methods (pages 24-26);

resolution (pages 26-27);

arrays and combinatorial arrays (pages 27-30);

software and calibration (pages 30-35; 68-70);

kits and other articles including tips coated with hydrophobic compounds (pages 35-37);

working examples (pages 38-67);

corresponding claims and abstract (pages 71-82); and

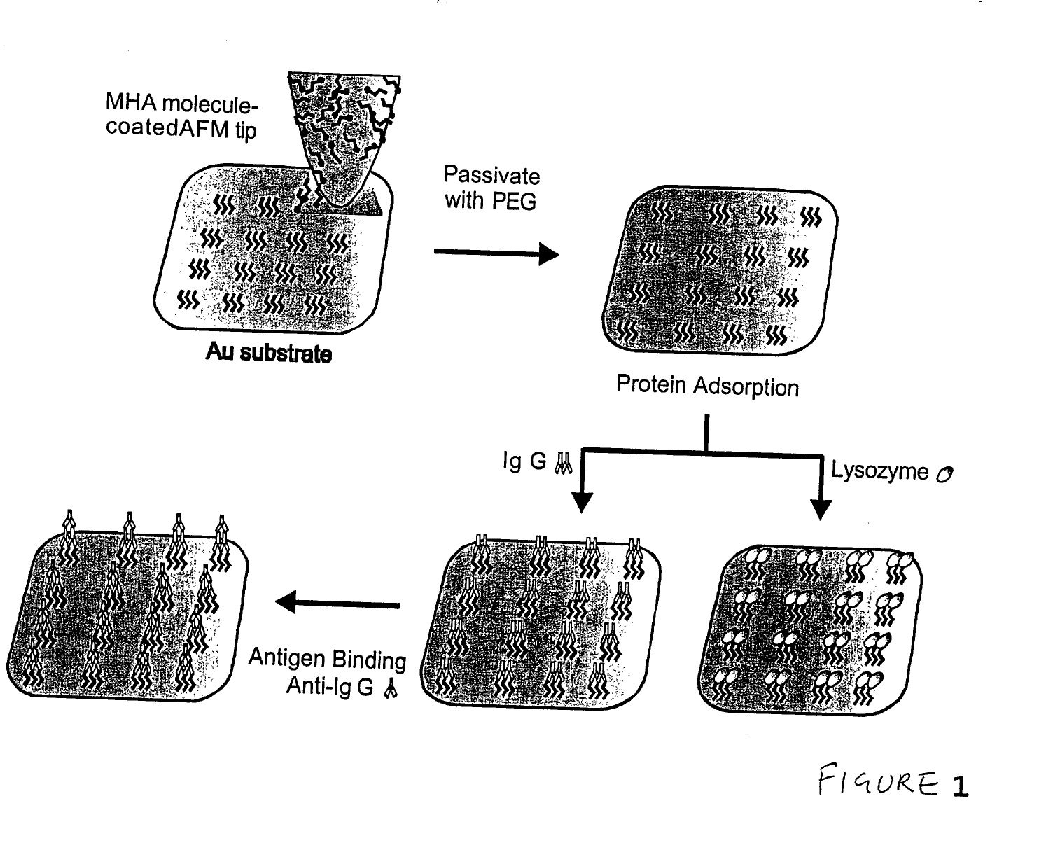

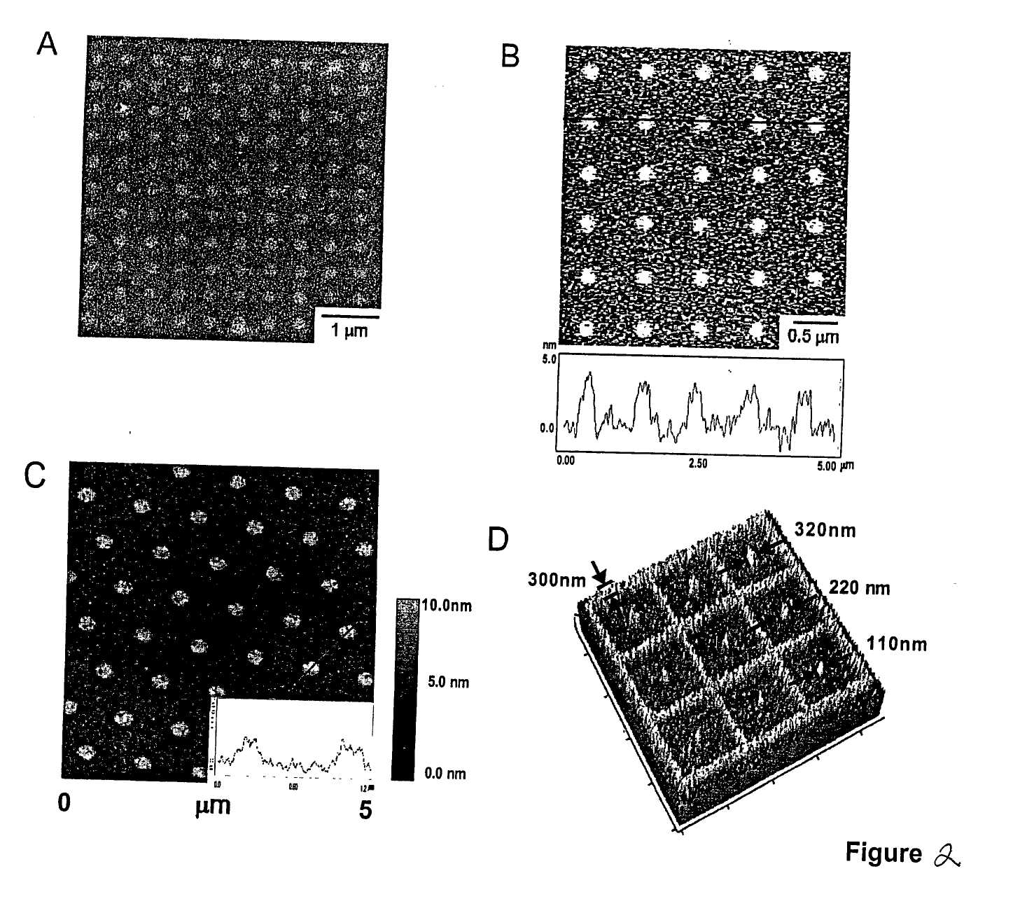

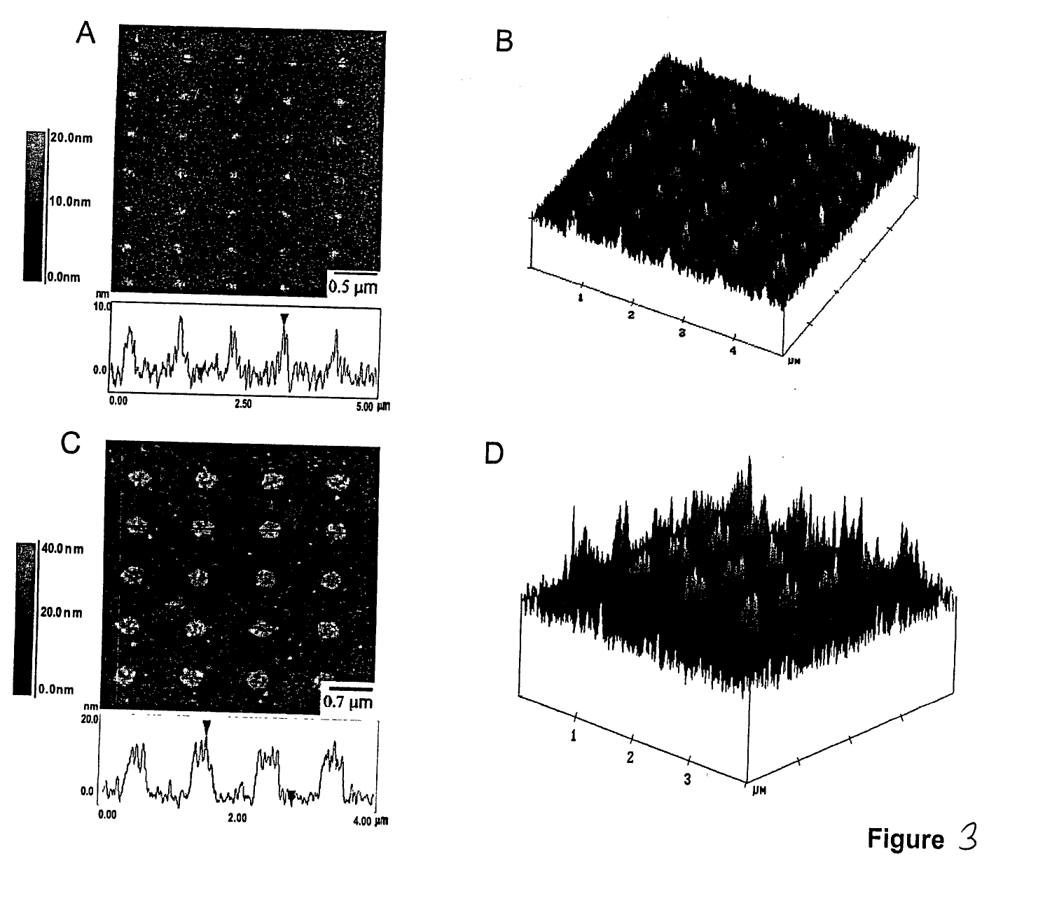

FIGS. 1-28.

All of the above priority document text, including each of the various subsections enumerated above including the figures, is hereby incorporated by reference in its entirety and form part of the present disclosure, supporting the claims.

DIP PEN™ nanolithographic printing, and the aforementioned procedures, instrumentation, and working examples, surprisingly can be adapted also to generate protein and peptide nanoarrays as described further herein. An approach generally used is illustrated in FIG. 1.

DIP PEN™ nanolithographic printing, particularly parallel DIP PEN™ nanolithographic printing, is also especially useful for the preparation of nanoarrays, particular combinatorial nanoarrays. An array is an arrangement of a plurality of discrete sample areas, or pattern units, forming a larger pattern on a substrate. The sample areas, or patterns, may be any shape (e.g., dots, lines, circles, squares or triangles) and may be arranged in any larger pattern (e.g., rows and columns, lattices, grids, etc. of discrete sample areas). Each sample area may contain the same or a different sample as contained in the other sample areas of the array. A “combinatorial array” is an array wherein each sample area or a small group of replicate sample areas (usually 2-4) contain(s) a sample which is different than that found in other sample areas of the array. A “sample” is a material or combination of materials to be studied, identified, reacted, etc.

DIP PEN™ nanolithographic printing, particularly parallel DIP PEN™ nanolithographic printing, is particularly useful for the preparation of nanoarrays and combinatorial nanoarrays on the submicrometer scale. An array on the submicrometer scale means that at least one of the dimensions (e.g, length, width or diameter) of the sample areas, excluding the depth, is less than 1 μm. DIP PEN™ nanolithographic printing, for example, can be used to prepare dots that are 10 nm in diameter. With improvements in tips (e.g., sharper tips), dots can be produced that approach 1 nm in diameter. Arrays on a submicrometer scale allow for faster reaction times and the use of less reagents than the currently-used microscale (i.e., having dimensions, other than depth, which are 1-999 μm) and larger arrays. Also, more information can be gained per unit area (i.e., the arrays are more dense than the currently-used micrometer scale arrays). Finally, the use of submicrometer arrays provides new opportunities for screening. For instance, such arrays can be screened with SPM's to look for physical changes in the patterns (e.g., shape, stickiness, height) and/or to identify chemicals present in the sample areas, including sequencing of nucleic acids.

Each sample area of an array can contain a single sample. For instance, the sample may be a biological material, such as a nucleic acid (e.g., an oligonucleotide, DNA, or RNA), protein or peptide (e.g., an antibody or an enzyme), ligand (e.g., an antigen, enzyme substrate, receptor or the ligand for a receptor), or a combination or mixture of biological materials (e.g., a mixture of proteins). Such materials may be deposited directly on a desired substrate as described above (see the description of patterning compounds noted above in the priority document). Alternatively, each sample area may contain a compound for capturing the biological material. See, e.g. PCT applications W000/04382, WO 00/04389 and WO 00/04390, the complete disclosures of which are incorporated herein-by reference. For instance, patterning compounds terminating in certain functional groups (e.g., —COOH) can bind proteins through a functional group present on, or added to, the protein (e.g., —NH₂). Also, it has been reported that polylysine, which can be attached to the substrate as described above, promotes the binding of cells to substrates. See James et al., Langmuir, 14, 741-744 (1998). As another example, each sample area may contain a chemical compound (organic, inorganic and composite materials) or a mixture of chemical compounds. Chemical compounds may be deposited directly on the substrate or may be attached through a functional group present on a patterning compound present in the sample area. As yet another example, each sample area may contain a type of microparticle or nanoparticle. See Example 7. From the foregoing, those skilled in the art will recognize that a patterning compound may comprise a sample or may be used to capture a sample.

The present invention is particularly focused on peptide and protein nanoarrays. Arrays and methods of using arrays are known in the art. For instance, such arrays can be used for biological and chemical screenings to identify and/or quantitate a biological or chemical material (e.g., immunoassays, enzyme activity assays, genomics, and proteomics). Biological and chemical libraries of naturally-occurring or synthetic compounds and other materials, including cells, can be used, e.g., to identify and design or refine drug candidates, enzyme inhibitors, ligands for receptors, and receptors for ligands, and in genomics and proteomics. Arrays of microparticles and nanoparticles can be used for a variety of purposes (see Example 7). Arrays can also be used for studies of crystallization, etching (see Example 5), etc. References describing combinatorial arrays and other arrays and their uses include U.S. Pat. Nos. 5,747,334, 5,962,736, and 5,985,356, and PCT applications WO 96/31625, WO 99/31267, WO 00/04382, WO 00/04389, WO 00/04390, WO 00/36 136, and WO 00/46406, which are hereby incorporated by reference in their entirety. Finally, results of experiments performed on the arrays of the invention can be detected by conventional means (e.g., fluorescence, chemiluminescence, bioluminescence, and radioactivity). Alternatively, an SPM can be used for screening arrays. For instance, an AFM can be used for quantitative imaging and identification of molecules, including the imaging and identification of chemical and biological molecules through the use of an SPM tip coated with a chemical or biomolecular identifier. See Frisbie et al., Science, 265,2071 2074 (1994); Wilbur et al., Langmuir, 11, 825-831 (1995); Noy et al., J. Am. Chem. Soc., 117, 7943-7951 (1995); Noy et al., Langmuir, 14, 1508-1511 (1998); and U.S. Pat. Nos. 5,363,697, 5,372,93, 5,472,881 and 5,874,668, the complete disclosures of which are incorporated herein by reference.

DIP PEN™ nanolithographic printing is particularly useful for the preparation of nanoarrays, arrays on the submicrometer scale having nanoscopic features. Preferably, a plurality of dots or a plurality of lines are formed on a substrate. The plurality of dots can be a lattice of dots including hexagonal or square lattices as known in the art. The plurality of lines can form a grid, including perpendicular and parallel arrangements of the lines.

The dimensions of the individual patterns including dot diameters and the line widths can be, for example, about 1,000 nm or less, about 500 nm or less, about 300 nm or less, and more particularly about 100 nm or less. The range in dimension can be for example about 1 nm to about 750 nm, about 10 nm to about 500 nm, and more particularly about 100 nm to about 350 nm.

The number of patterns in the plurality of patterns is not particularly limited. It can be, for example, at least 10, at least 100, at least 1,000, at least 10,000, even at least 100,000. Square arrangements are possible such as, for example, a 10×10 array. High density arrays are preferred.

The distance between the individual patterns on the nanoarray can vary and is not particularly limited. For example, the patterns can be separated by distances of less than one micron or more than one micron. The distance can be, for example, about 300 to about 1,500 microns, or about 500 microns to about 1,000 microns. Distance between separated patterns can be measured from the center of the pattern such as the center of a dot or the middle of a line.

In the peptide and protein nanoarrays of this invention, the nanoarrays can be prepared comprising various kinds of chemical structures comprising peptide bonds. These include peptides, proteins, oligopeptides, and polypeptides, be they simple or complex. The peptide unit can be in combination with non-peptide units. The protein or peptide can contain a single polypeptide chain or multiple polypeptide chains. Higher molecular weight peptides are preferred in general although lower molecular weight peptides including oligopeptides can be used. The number of peptide bonds in the peptide can be, for example, at least three, ten or less, at least 100, about 100 to about 300, or at least 500.

Proteins are particularly preferred. The protein can be simple or conjugated. Examples of conjugated proteins include, but are not limited to, nucleoproteins, lipoproteins, phosphoproteins, metalloproteins and glycoproteins.

Proteins can be functional when they coexist in a complex with other proteins, polypeptides or peptides. The protein can be a virus, which can be complexes of proteins and nucleic acids, be they of the DNA or RNA types. The protein can be a shell to larger structures such as spheres and rod structures.

Proteins can be globular or fibrous in conformation. The latter are generally tough materials that are typically insoluble in water. They can comprise a polypeptide chain or chains arranged in parallel as in, for example, a fiber. Examples include collagen and elastin. Globular proteins are polypeptides that are tightly folded into spherical or globular shapes and are mostly soluble in aqueous systems. Many enzymes, for instance, are globular proteins, as are antibodies, some hormones and transport proteins, like serum albumin and hemoglobin.

Proteins can be used which have both fibrous and globular properties, like myosin and fibrinogen, which are tough, rod-like structures but are soluble. The proteins can possess more than one polypeptide chain, and can be oligomeric proteins, their individual components being called protomers. The oligomeric proteins usually contain an even number of polypeptide chains, not normally covalently linked to one another. Hemoglobin is an example of an oligomeric protein.

Types of proteins that can be incorporated into a nanoarray of the present invention include, but are not limited to, enzymes, storage proteins, transport proteins, contractile proteins, protective proteins, toxins, hormones and structural proteins.

Examples of enzymes include, but are not limited to ribonucleases, cytochrome c, lysozymes, proteases, kinases, polymerases, exonucleases and endonucleases. Enzymes and their binding mechanisms are disclosed, for example, in Enzyme Structure and Mechanism, 2^{nd} Ed., by Alan Fersht, 1977 including in Chapter 15 the following enzyme types: dehydrogenases, proteases, ribonucleases, staphyloccal nucleases, lysozymes, carbonic anhydrases, and triosephosphate isomerase.

Examples of storage proteins include, but are not limited to ovalbumin, casein, ferritin, gliadin, and zein.

Examples of transport proteins include, but are not limited to hemoglobin, hemocyanin, myoglobin, serum albumin, β1-lipoprotein, iron-binding globulin, ceruloplasmin.

Examples of contractile proteins include, but are not limited to myosin, actin, dynein.

Examples of protective proteins include, but are not limited to antibodies, complement proteins, fibrinogen and thrombin.

Examples of toxins include, but are not limited to, Clostridium botulinum toxin, diptheria toxin, snake venoms and ricin.

Examples of hormones include, but are not limited to, insulin, adrenocorticotrophic hormone and insulin-like growth hormone, and growth hormone.

Examples of structural proteins include, but are not limited to, viral-coat proteins, glycoproteins, membrane-structure proteins, α-keratin, sclerotin, fibroin, collagen, elastin and mucoproteins.

Natural or synthetic peptides and proteins can be used. Proteins can be used, for example, which are prepared by recombinant methods.

Examples of preferred proteins include immunoglobulins, IgG (rabbit, human, mouse, and the like), Protein A/G, fibrinogen, fibronectin, lysozymes, streptavidin, avdin, ferritin, lectin (Con. A), and BSA. Rabbit IgG and rabbit anti-IgG, bound in sandwhich configuration to IgG are useful examples.

Spliceosomes and ribozomes and the like can be used.

A wide variety of proteins are known to those of skill in the art and can be used. See, for instance, Chapter 3, “Proteins and their Biological Functions: A Survey,” at pages 55-66 of BIOCHEMISTRY by A. L. Lehninger, 1970, which is incorporated herein by reference.

A variety of peptide type compounds, including proteins, polypeptides, and oligopeptides can be directly transferred and adsorbed to surfaces in a patterned fashion with use of DIP PEN™ nanolithographic printing, wherein the peptide or protein is directly transferred from a tip such as, an atomic force microscope tip, to a substrate. Alternatively, however, in an indirect method, the DIP PEN™ nanolithographic printing can be used to deposit or deliver a compound in a pattern (a patterning compound), and then the peptide or protein can be assembled onto or adsorbed to the patterning compound after patterning.

The methods described in the incorporated priority document (09/866,533), known in the art, can be used and need not be repeated in their entirety here. For example, known substrates and known patterning compounds can be used to make nanoarrays. Smoother substrates are generally preferred which provide for high resolution printing.

For example, a nanoarray substrate having a nanoarray surface can be, for example, an insulator such as, for example, glass or a conductor such as, for example, metal, including gold. In addition, the substrate can be a metal, a semiconductor, a magnetic material, a polymer material, a polymer-coated substrate, or a superconductor material. The substrate can be previously treated with one or more adsorbates. Still further, examples of suitable substrates include but are not limited to, metals, ceramics, metal oxides, semiconductor materials, magnetic materials, polymers or polymer coated substrates, superconductor materials, polystyrene, and glass. Metals include, but are not limited to gold, silver, aluminum, copper, platinum and palladium. Other substrates onto which compounds may be patterned include, but are not limited to silica, silicon oxide, GaAs, and InP.

The patterning compound can be chemisorbed or covalently bound to the substrate to anchor the patterning compound and improve stability. It can be, for example, a sulfur-containing compound such as, for example, a thiol, polythiol, sulfide, cyclic disulfide, and the like. It can be, for example, a sulfur-containing compound having a sulfur group at one end and a terminal reactive group at the other end, such as an alkane thiol with a carboxylic acid end group. The patterning compound can be a lower molecular weight compound of less than, for example, 100, or less than 500, or less than 1,000, or a higher molecular weight compound including oligomeric and polymeric compounds. Synthetic and natural patterning compounds can be used. Other examples include alkanethiols that have functional end-groups such as 16-mercaptohexadecanoic acid; hydrophobic thiols, such as 1 -octadecanethiol; and organic coupling molecules, such as EDC and mannose-SH. Other examples of sulfur-containing compounds include, but are not limited to, hydrogen sulphide, mercaptans, thiols, sulphides, thioesters, polysulphides, cyclic sulphides, and thiophene derivatives. For instance, a sulfur-containing compound may comprise a thiol, phosphothiol, thiocyano, sulfonic acid, disulfide or isothiocyano group.

Other compounds include silicon-containing compounds that have a siloxy or silyl group that posseses a carboxylic acid group, aldehydes, alcohol, alkoxy or vinyl group. A compound may also possess an amine, nitrile, or isonitrile group.

Sulfur adsorption on gold is a preferred system, but the invention is not limited to this embodiment.

In general, therefore, the inventive method involves using nanolithographic methods, preferably DIP PEN™ nanolithographic printing, to deposit a compound onto a surface to produce a “preformed array template,” and then assembling onto that surface, peptides and proteins that adsorb to those compounds. The “assembling” process may be achieved by exposing the preformed array template to a solution containing the desired peptide or protein, i.e., the inventive method can comprise immersing a preformed array template into a peptide or protein solution; or spraying the solution onto the surface of the preformed array template. Other methods of exposing the preformed array template to a peptide or protein solution include placing the array in a chamber containing a peptide or protein solution vapor or mist, or pouring the peptide or protein solution onto the template. Alternatively, the assembling process may include depositing the peptide or protein onto a compound of the preformed array template using DIP PEN™ nanolithographic printing.

Non-specific binding of proteins to other, “non-compound” regions of a surface, can be prevented by covering, or “passivating,” those regions of the surface with another compound, or mixture of compounds, prior to exposure to the protein solution or sample (one or more passivating compounds). Known passivating compounds can be used and the invention is not particularly limited by this feature to the extent that non-specific adsorption does not occur. A variety of passivating compounds can be used including, for example, surfactants such as alkylene glycols which are functionalized to adsorb to the substrate. An example of a compound useful for passivating is 11-mercaptoundecyl-tri(ethylene glycol). Proteins can have a relatively weak affinity for surfaces coated with 11-mercaptoundecyl-tri(ethylene glycol) and therefore do not bind to such surfaces. See, for instance, Browning-Kelley et al., Langmuir 13, 343, 1997; Waud-Mesthrige et al., Langmuir 15, 8580, 1999; Waud-Mesthrige et al., Biophys. J. 80 1891, 2001; Kenseth et al., Langmuir 17, 4105, 2001; Prime & Whitesides, Science 252, 1164, 1991; and Lopez et al., J.Am.Chem.Soc. 115, 10774, 1993, which are hereby incorporated by reference. However, other chemicals and compounds, such as bovine serum albumin (BSA) and powdered milk, that can be used to cover a surface in similar fashion to prevent non-specific binding of proteins to a surface. BSA, however, can provide less performance than 11-mercaptoundecyl-tri(ethylene glycol). After passivation, the resultant array can be called a passivated array of proteins or peptides. Alternatively, the DIP PEN™ nanolithographic printing method can be used to pattern a passivating compound, and peptide and protein adsorption can be carried out on the other non-passivated areas.

The invention is not particularly limited by the type of interaction between the peptide or protein and the patterning compound. In general, its preferred that the interaction results in a functionally useful protein after absorption and that the interaction is strong. Compound-protein bonds can be by, for example, covalent, ionic, hydrogen bonding, or electrostatic interactions. Thus, a covalent bond can be formed between a protein and a compound that is deposited onto a surface. Such compounds include, but are not limited to, terminal succinimide groups, aldehyde groups, carboxyl groups and photoactivatable aryl azide groups. Furthermore, the spontaneous coupling of succinimide, or in the alternative, aldehyde surface groups, to primary amines in a protein at a physiological pH may be incorporated for attaching proteins to the surface. For instance, proteins often have a high affinity for carboxilic acid terminated monolayers at pH 7, such as those exhibited by 16-mercaptohexadecanoic acid (“MHA”). Photoactivatable surfaces, such as those containing aryl azides, may also be used to bind proteins. Thus, photoactivatable surfaces form highly reactive nitrenes that react with a variety of chemical groups upon ultraviolet activation.

One may also modify array components to exploit interactions between various biochemical moieties that may not naturally occur. For example, histidine binds tightly to nickel. Therefore, proteins modified using recombinant methods to produce stretches of histidine residues, usually 6 to 10 amino acids long, could bind to nickel-containing compounds deposited onto a surface. Alternatively, sulfhydryl groups can be introduced into proteins, or they may be naturally occurring in the protein, and used to bind proteins to compounds already bound onto a gold surface. Similarly, a compound may be modified so as to comprise a sulfhydryl group. The compound can then bind to a gold surface and also bind to a protein.

The protein that binds to the compound deposited on the surface of the array may itself bind a variety of targets, including protein targets, i.e., other “target proteins” and/or perform or elicit biological or chemical reactivity, such as enzyme catalysis, cleavage or hydrolysis. Thus, according to the invention, a protein that is adsorbed to a surface via a compound deposited onto that surface may be used to, for example, (i) bind a target, (ii) react and utilize a substrate, or (iii) be used as a substrate for utilization by a target.

For instance, the atomic force microscopy (AFM) can be employed to screen arrays of the present invention to provide information, such as protein reactivity, at the single-protein level, or to detect binding of a target such as a target protein to a protein in an array. For example, the height, hydrophobicity, stickiness, roughness, and shape of the location where the capture protein is bound most likely will change upon reaction with or binding to another substance. All of such variables are easily probed with a conventional atomic force microscope. Other probe or detection methods can also be used as known to those skilled in the art.

A nanoscopic protein array, or nanoarray, of the present invention can be useful for a wide variety of technological applications, such as for example proteomics; pharmacological research; performing immunoassays; investigating protein-protein interactions; and determining levels, amounts or concentrations of specific substances in a sample. They can be useful in biology to study cell control and guidance; and they also are useful in information technology. With respect to the latter, ordered biomolecular arrays can be tailored to make ultrahigh-density, nanometer-scale bioelectronic integrated circuits.

In one specific embodiment, illustrated in FIGS. 1-5 and working Example 8 below, nanoscopic lysozyme and rabbit immunoglobulin G (“IgG”) nanoarrays were made according to the inventive techniques. DIP PEN™ nanolithographic printing was used to pattern the compound, 16-mercaptohexadecanoic acid, onto the surface of a gold film, in the form of dots or lined grids. The areas surrounding the MHA dots or lines were then passivated with 11-mercaptoundecyl-tri(ethylene glycol), a surfactant. The patterned and passivated gold film was then immersed in either a solution containing lysozyme of rabbit IgG and then rinsed. The protein arrays were then characterized by AFM, which showed that lysozyme proteins assembled only on the MHA-patterned surfaces of the gold film to form an array of dots or lines. Since lysozyme is ellipsoidal in shape, it can adopt at two significantly different conformations (i.e., lying on its long axis or standing upright) on the gold film surface. Both of these conformations could be differentiated by measuring differences in height by AFM.

Similarly, rabbit IgG was measured according to height statistics once it was bound to the gold film surface. Like the lysozyme array, the rabbit IgG only bound to the nanoscopic MHA pattern. The bioactivity of the MHA-bound IgG immunoglobulins was evaluated by testing the reactivity of the IgG with an anti-IgG protein which is known to form a strongly bound complex with IgG. It was found that the anti-IgG only bound to the IgG, resulting in an increase in height, measurable by AFM. Thus, detecting a change in height (i.e., before and after exposure to anti-IgG) proves an easy way of screening the array for positive signals. A simultaneously-conducted control experiment is useful to show that binding of, in this case, anti-IgG to IgG, is not random or non-specific. For instance, no anti-IgG proteins became bound to the lysozyme array described above, as was evidenced by a lack of change in lysozyme height profile. See, for example, Lee et al., Science, 295, pp.1702-1705, 2002.

The resolution of the methods described herein can be evaluated and optimized, and integrated nanolibraries of proteins can be made.

The invention is further illustrated by the following working Examples. In particular, Example 8 focuses on peptide and protein nanoarrays. Examples 1-7 illustrate various embodiments for DIP PEN™ nanolithographic printing.

### EXAMPLES

### Example 1

### DIP PEN™ Nanolithographic Printing with Alkanethiols on A Gold Substrate

When an AFM tip coated with ODT is brought into contact with a sample surface, the ODT flows from the tip to the sample by capillary action, much like a dip pen. This process has been studied using a conventional AFM tip on thin film substrates that were prepared by thermally evaporating 300 Å of polycrystalline Au onto mica at room temperature. A Park Scientific Model CP AFM instrument was used to perform all experiments. The scanner was enclosed in a glass isolation chamber, and the relative humidity was measured with a hygrometer. All humidity measurements have an absolute error of ±5%. A silicon nitride tip (Park Scientific, Microlever A) was coated with ODT by dipping the cantilever into a saturated solution of ODT in acetonitrile for 1 minute. The cantilever was blown dry with compressed difluoroethane prior to use.

A simple demonstration of the DIP PEN™ nanolithographic™ printing process involved raster scanning a tip that was prepared in this manner across a 1 μm by 1 μm section of a Au substrate. An LFM image of this section within a larger scan area (3 μm by 3 μm) showed two areas of differing contrast. The interior dark area, or region of lower lateral force, was a deposited monolayer of ODT, and the exterior lighter area was bare Au.

Formation of high-quality self-assembled monolayers (SAMs) occurred when the deposition process was carried out on Au(111)/mica, which was prepared by annealing the Au thin film substrates at 300° C. for 3 hours. Alves et al., J Am. Chem. Soc., 114:1222 (1992). In this case, it was possible to obtain a lattice-resolved image of an ODT SAM. The hexagonal lattice parameter of 5.0±0.2 Å compares well with reported values for SAMs of ODT on Au(111) (Id.) and shows that ODT, rather than some other adsorbate (water or acetonitrile), was transported from the tip to the substrate.

Although the experiments performed on Au(111)/mica provided important information about the chemical identity of the transported species in these experiments, Au(l111)/mica is a poor substrate for DIP PEN™ nanolithographic printing. The deep valleys around the small Au(111) facets make it difficult to draw long (micrometer) contiguous lines with nanometer widths.

The nonannealed Au substrates are relatively rough (root-mean square roughness 2 nm), but 30 nm lines could be deposited by DIP PEN™ nanolithographic printing. This distance is the average Au grain diameter of the thin film substrates and represents the resolution limit of DIP PEN™ nanolithographic™ printing on this type of substrate. The 30-nm molecule-based line prepared on this type of substrate was discontinuous and followed the grain edges of the Au. Smoother and more contiguous lines could be drawn by increasing the line width to 100 nm or presumably by using a smoother Au substrate. The width of the line depends upon tip scan speed and rate of transport of the alkanethiol from the tip to the substrate (relative humidity can change the transport rate). Faster scan speeds and a smaller number of traces give narrower lines.

DIP PEN™ nanolithographic printing was also used to prepare molecular dot features to demonstrate the diffusion properties of the “ink”. The ODT-coated tip was brought into contact (set point=1 nN) with the Au substrate for a set period of time. For example, 0.66 μm, 0.88 μm, and 1.6 μm diameter ODT dots were generated by holding the tip in contact with the surface for 2, 4, and 16 minutes, respectively. The uniform appearance of the dots likely reflects an even flow of ODT in all directions from the tip to the surface. Opposite contrast images were obtained by depositing dots of an alkanethiol derivative, 16-mercaptohexadecanoic acid in an analogous fashion. This not only provides additional evidence that the molecules are being transported from the tip to the surface but also demonstrates the molecular generality of DIP PEN™ nanolithographic printing.

Arrays and grids could be generated in addition to individual lines and dots. An array of twenty-five 0.46-μm diameter ODT dots spaced 0.54 μm apart was generated by holding an ODT-coated tip in contact with the surface (1 nM) for 20 seconds at 45% relative humidity without lateral movement to form each dot. A grid consisting of eight intersecting lines 2 μm in length and 100 nm wide was generated by sweeping the ODT-coated tip on a Au surface at a 4 μm per second scan speed with a 1 nN force for 1.5 minutes to form each line.

### Example 2

### DIP PEN™ Nanolithographic Printing With A Variety of Substrates And “Inks”

A large number of compounds and substrates have been successfully utilized in DIP PEN™ nanolithographic printing. They are listed below in Table 1, along with possible uses for the combinations of compounds and substrates.

AFM tips (Park Scientific) were used. The tips were silicon tips, silicon nitride tips, and silicon nitride tips coated with a 10 nm layer of titanium to enhance physisorption of patterning compounds. The silicon nitride tips were coated with the titanium by vacuum deposition as described in Holland, Vacuum Deposition Of Thin Films (Wiley, New York, N.Y., 1956). It should be noted that coating the silicon nitride tips with titanium made the tips dull and decreased the resolution of DIP PEN™ nanolithographic printing. However, titanium-coated tips are useful when water is used as the solvent for a patterning compound. DIP PEN™ nanolithographic printing performed with uncoated silicon nitride tips gave the best resolution (as low as about 10 nm).

Metal film substrates listed in Table 1 were prepared by vacuum deposition as described in Holland, Vacuum Deposition Of Thin Films (Wiley, New York, N.Y., 1956). Semiconductor substrates were obtained from Electronic Materials, Inc., Silicon Quest, Inc. MEMS Technology Applications Center, Inc., or Crystal Specialties, Inc.

The patterning compounds listed in Table 1 were obtained from Aldrich Chemical Co. The solvents listed in Table 1 were obtained from Fisher Scientific.

The AFM tips were coated with the patterning compounds as described in Example 1 (dipping in a solution of the patterning compound followed by drying with an inert gas), by vapor deposition or by direct contact scanning. The method of Example 1 gave the best results. Also, dipping and drying the tips multiple times further improved results.

The tips were coated by vapor deposition as described in Sherman, Chemical Vapor Deposition For Microelectronics: Principles, Technology And Applications (Noyes, Park Ridges, N.J., 1987). Briefly, a patterning compound in pure form (solid or liquid, no solvent) was placed on a solid substrate (e.g., glass or silicon nitride; obtained from Fisher Scientific or MEMS Technology Application Center) in a closed chamber. For compounds which are oxidized by air, a vacuum chamber or a nitrogen-filled chamber was used. The AFM tip was position about 1-20 cm from the patterning compound, the distance depending on the amount of material and the chamber design. The compound was then heated to a temperature at which it vaporizes, thereby coating the tip with the compound. For instance, 1-octadecanethiol can be vapor deposited at 60° C. Coating the tips by vapor deposition produced thin, uniform layers of patterning compounds on the tips and gave quite reliable results for DIP PEN™ nanolithographic printing.

The tips were coated by direct contact scanning by depositing a drop of a saturated solution of the patterning compound on a solid substrate (e.g., glass or silicon nitride; obtained from Fisher Scientific or MEMS Technology Application Center). Upon drying, the patterning compound formed a microcrystalline phase on the substrate. To load the patterning compound on the AFM tip, the tip was scanned repeatedly (−5Hz scan speed) across this microcrystalline phase. While this method was simple, it did not lead to the best loading of the tip, since it was difficult to control the amount of patterning compound transferred from the substrate to the tip.

DIP PEN™ nanolithographic printing was performed as described in Example 1 using a Park Scientific AFM, Model CP, scanning speed 5-10 Hz. Scanning times ranged from 10 seconds to 5 minutes. Patterns prepared included grids, dots, letters, and rectangles. The width of the grid lines and the lines that formed the letters ranged from 15 nm to 250 nm, and the diameters of the individual dots ranged from 12 nm to 5 micrometers.

TABLE 1

| | Patterning | Potential | |
|---|---|---|---|
| Substrate | Compound/Solvent(s) | Applications | Comments and References |
| Au | n-octadecanethiol/ | Basic research | Study of intermolecular forces, |
| | acetonitrile, ethanol | | Langmuir 10, 3315 (1994) |
| | | Etching resist for | Etchant: KCN/O₂(pH˜14), |
| | | microfabrication | J. Vac. Sci. Tech. B, 13, 1139 (1995) |
| | Dodecanethiol/ | Molecular | Insulating thin coating on nanometer |
| | acetonitrile, ethanol | electronics | scale gold clusters. |
| | | | Superlattices and Microstructures 18, |
| | | | 275 (1995) |
| | n-hexadecanethiol/ | Etching resist for | Etchant: KCN/O₂(pH˜14). |
| | acetonitrile, ethanol | microfabrication | Langmuir, 15, 300 (1999) |
| | n-docosanethiol/acetonitrile, | Etching resist for | Etchant: KCN/O₂(pH˜14). |
| | ethanol | microfabrication | J. Vac. Sci. Technol. B, 13, 2846 (1995) |
| | 11-mercapto-1- | Surface | Capturing SiO₂ clusters |
| | undecanol/ | functionalization | |
| | acetonitrile, ethanol | | |
| | 16-mercapto-1- | Basic research | Study of Intermolecular forces. |
| | hexadecanoic acid/ | | Langmuir 14, 1508 (1998) |
| | acetonitrile, ethanol. | | |
| | | Surface | Capturing SiO₂, SnO₂ clusters. |
| | | functionalization | J. Am. Chem. Soc., 114, 5221 (1992) |
| | Octanedithiol/ | Basic research | Study of intermolecular forces. |
| | acetonitrile, ethanol | | Jpn. J. Appi. Phys. 37, L299 (1998) |
| | Hexanedithiol/ | Surface | Capturing gold clusters. J. Am. |
| | acetonitrile, ethanol | functionalization | Chem. Soc., 114, 5221 (1992) |
| | Propanedithiol/ | Basic research | Study of intermolecular forces. J. |
| | acetonitrile, ethanol | | Am. Chem. Soc., 114, 5221 (1992) |
| | α,α′-ρ-xylyldithiol/ | Surface | Capturing gold clusters. |
| | acetonitrile, ethanol | functionalization | Science, 272, 1323 (1996) |
| | | Molecular | Conducting nanometer scale junction |
| | | electronics | Science, 272, 1323 (1996) |
| | 4,4′-biphenyldithiol/ | Surface | Capturing gold and CdS clusters, |
| | acetonitrile, ethanol | functionalization | Inorganica Chemica Acta 242, |
| | | 115 (1996) | |
| | Terphenyldithiol/ | Surface | Capturing gold and CdS clusters, |
| | acetonitrile, ethanol | functionalization | Inorganica Chemica Acta 242, |
| | | 115 (1996) | |
| | terphenyldiisocyanide/ | Surface | Capturing gold and CdS clusters, |
| | acetonitrile, | functionalization | Inorganica Chemica Acta 242, |
| | methylene chloride | | 115 (1996) |
| | | Molecular | Conductive coating on nanometer scale |
| | | electronics | gold clusters. Superlattices and |
| | | | Microstructures, 18, 275 (1995) |
| | DNA/ | Gene detection | DNA probe to detect biological cells. |
| | water: acetonitrile (1.3) | | J. Am. Chem. Soc. 119, 8916 (1997) |
| Ag | n-hexadecanethiol/ | Etching resist for | Etchant: Fe(NO₃)₃(pH˜6). |
| | acetonitrile, ethanol | microfabrication | Microlectron. Eng., 32, 255 (1996) |
| Al | 2-mercaptoacetic acid/ | Surface | Capturing CdS clusters. |
| | acetonitrile, ethanol | functionalization | J. Am. Chem. Soc., 114, 5221 (1992) |
| GaAs-100 | n-octadecanethiol/ | Basic research | Self assembled monolayer formation |
| | acetonitrile, ethanol | Etching resist for | HCl/HNO₃(pH˜1). |
| | | microfabrication | J. Vac. Sci. Technol. B, 11, 2823 (1993) |
| TiO2 | n-octadecanethiol/ | Etching resist for | |
| | acetonitrile, ethanol | microfabrication | |
| SiO2 | 16-mercapto-1-hexadecanoic | Surface | Capturing gold and CdS clusters |
| | acid/acetonitrile, | functionalization | |
| | ethanol | | |
| | octadecyltrichlorosila | Etching resist for | Etchant: HF/NH₄F (pH˜2). |
| | ne(OTS, | microfabrication | Appl. Phys. Lett., 70, 1593 (1997) |
| | CH₃(CH₂)₁₇SiCl₃) | | |
| | 1.2nm thick SAM/ | | |
| | hexane | | |
| | APTS, 3-(2- | Surface | Capturing nanometer scale gold |
| | Aminoethylamino)pro | functionalization | clusters |
| | pyltrimethoxysilane/ | | Appl. Phys. Lett. 70, 2759 (1997) |
| | water | | |

### Example 3

### Atomic Force Microscopy With Coated Tips

This example describes the modification of silicon nitride AFM tips with a physisorbed layer of 1-dodecylamine. Such tips improve one's ability to do LFM in air by substantially decreasing the capillary force and providing higher resolution, especially with soft materials.

All data presented in this example were obtained with a Park Scientific Model CP AFM with a combined AFM/LFM head. Cantilevers (model no. MLCT-AUNM) were obtained from Park Scientific and had the following specifications: gold coated microlever, silicon nitride tip, cantilever A, spring constant=0.05N/m. The AFM was mounted in a Park vibration isolation chamber which had been modified with a dry nitrogen purge line. Also, an electronic hygrometer, placed inside the chamber, was used for humidity measurements (±5% with a range of 12˜100%). Muscovite green mica was obtained from Ted Pella, Inc. Soda lime glass microscope slides were obtained from Fisher. Polystyrene spheres with 0.23±0.002 μm diameters were purchased from Polysciences, and Si₃N₄ on silicon was obtained from MCNC MEMS Technology Applications Center. 1 -Dodecylamine (99+%) was purchased from Aldrich Chemical Inc. and used without further purification. Acetonitrile (A.C.S. grade) was purchased from Fisher Scientific Instruments, Inc.

Two methods for coating an AFM tip with 1-dodecylamine were explored. The first method involved saturating ethanol or acetonitiile with 1-dodecylamine and then depositing a droplet of this solution on a glass substrate. Upon drying, the 1-dodecylamine formed a microcrystalline phase on the glass substrate. To load the 1-dodecylamine on the AFM tip, the tip was scanned repeatedly (˜5Hz scan speed) across this microcrystalline phase. While this method was simple, it did not lead to the best loading of the tip, since it was difficult to control the amount of 1-dodecylamine transferred from the substrate to the tip.

A better method was to transfer the dodecylamine directly from solution to the AFM cantilever. This method involved soaking the AFM cantilever and tip in acetonitrile for several minutes in oMer to remove any residual contaminants on the tip. Then the tip was soaked in a ˜5 mM 1-dodecylamine/acetonitrile solution for approximately 30 seconds. Next, the tip was blown dry with compressed freon. Repeating this procedure several times typically gave the best results. The 1-dodecylamine is physisorbed, rather than chemisorbed, onto the silicon nitride tips. Indeed, the dodecylamine can be rinsed off the tip with acetonitrile as is the case with bulk silicon nitride. Benoit et al. Microbeam and Nanobeam Analysis; Springer Verlag, (1996). Modification of the tip in this manner significantly reduced the capillary effects due to atmospheric water condensation as evidenced by several experiments described below.

First, a digital oscilloscope, directly connected to the lateral force detector of the AFM, was used to record the lateral force output as a function of time. In this experiment, the force of friction changed direction when the tip scanned left to right, as compared with right to left. Therefore, the output of the LFM detector switched polarity each time the tip scan direction changed. If one or more AFM raster scans were recorded, the output of the detector was in the form of a square wave. The height of the square wave is directly proportional to the sliding friction of the tip on the sample and, therefore, one can compare the forces of friction between an unmodified tip and a glass substrate and between a modified tip and a glass substrate simply by comparing the height of the square waves under nearly identical scanning and environmental conditions. The tip/sample frictional force was at least a factor of three less for the modified tip than for the unmodified tip. This experiment was repeated on a mica substrate, and a similar reduction in friction was observed. In general, reductions in friction measured in this way and under these conditions ranged from a factor of three to more than a factor of ten less for the modified tips, depending upon substrate and environmental conditions, such as relative humidity.

While this experiment showed that 1-dodecylamine treatment of an AFM tip lowered friction, it did not prove that water and the capillary force were the key factors. In another experiment, the effects of the 1-dodecylamine coating on the capillary transport of water was examined. Details of water transport involving unmodified tips have been discussed elsewhere. Finer et al., Langmuir 13, 6864-6868 (1997). When an AFM tip was scanned across a sample, it transported water to the sample by capillary action. After scanning a 4 μm×5 μm area of a soda glass substrate for several minutes, contiguous adlayers of water were deposited onto the substrate and imaged by LFM by increasing the scan size. Areas of lower friction, where water had been deposited, appeared darker than non-painted areas. The same experiment conducted with a tip coated with 1-dodecylamine did not show evidence of substantial water transport. Indeed, only random variations in friction were observed.

While these experiments showed that friction could be reduced and the transport of water from the tip to the substrate by capillary action could be inhibited by coating the tip with 1-dodecylamine, they did not provide information about the resolving power of the modified tip. Mica is an excellent substrate to evaluate this issue and, indeed, lattice resolved images could be routinely obtained with the modified tips, demonstrating that this modification procedure reduced the force of friction without blunting the tip. It was difficult to determine whether the portion of the tip that was involved in the imaging was bare or had a layer of 1-dodecylamine on it. In fact, it is likely that the 1-dodecylamine layer had been mechanically removed from this part of the tip exposing the bare Si₃N_{4.} In any event, the remainder of the tip must have had a hydrophobic layer of dodecylamine on it, since water was inhibited from filling the capillary surrounding the point of contact, thereby reducing the capillary effect (see above).

While the atomic scale imaging ability of the AFM was not adversely affected by the 1-dodecyl amine coating on the tip, the above experiment did not provide useful information about the suitability of the tip for obtaining morphology data on a larger scale. In order to obtain such information, a sample of monodisperse 0.23 μm diameter latex spheres was imaged with both modified and unmodified tips. Since the topography recorded by an AFM is a convolution of the shape of the tip and the shape of the sample, any change in the shape of the tip will be reflected in a change in the imaged topography of the latex spheres. No detectable difference was found in images taken with unmodified and modified tips, respectively. This shows that the shape of the tip was not significantly changed as it would be if a metallic coating had been evaporated onto it. Moreover, it suggests that the 1-dodecylamine coating was fairly uniform over the surface of the tip and was sharp enough that it did not adversely affect atomic scale imaging.

A significant issue pertains to the performance of the modified tips in the imaging of soft materials. Typically, it is difficult to determine whether or not a chemically-modified tip exhibits improved performance as compared with a bare tip. This is because chemical modification is often an irreversible process which sometimes requires the deposition of an intermediary layer. However, since the modification process reported herein was based upon physisorbed layers of 1-dodecylamine, it was possible to compare the performance of a tip before modification, after modification, and after the tip had been rinsed and the 1-dodecylamine had been removed. Qualitatively, the 1-dodecylamine-modified tips always provided significant improvements in the imaging of monolayers based upon alkanethiols and organic crystals deposited onto a variety of substrates. For example, a lattice resolved image of a hydrophilic self-assembled monolayer of 11-mercapto-1-undecanol on a Au(111) surface was routinely obtained with a modified tip. The lattice could not be resolved with the same unmodified AFM tip. On this surface, the coated tip showed a reduction in friction of at least a factor of five by the square wave analysis (see above). It should be noted, that the OH-terminated SAM is hydrophilic and, hence, has a strong capillary attraction to a clean tip. Reducing the capillary force by the modified tip allows one to image the lattice.

A second example of improved resolution involved imaging free standing liquid surfaces, such as water condensed on mica. It is well known that at humidities between 30 and 40 percent, water has two distinct phases on mica. Hu et al., Science 268, 267-269 (1995). In previous work by this group, a non-contact mode scanning polarization force microscope (SPFM) was used to image these phases. It was found that, when a probe tip came into contact with mica, strong capillary forces caused water to wet the tip and strongly disturbed the water condensate on the mica. To reduce the capillary effect so that two phases of water could be imaged, the tip was kept ˜20 nm away from the surface. Because of this constraint, one cannot image such phases with a contact mode scanning probe technique. Images were obtained of the two phases of water on mica recorded at 30 percent humidity with a 1-dodecylamine modified tip in contact mode. The heights of the features corresponded with the frictional map, with higher features having lower friction. The quality of the modified tip, which it is believed correlates with the uniformity of the 1-dodecylamine layer on the tip, was important. Only well modified tips made it possible to image the two-phases of water, while less well modified ones resulted in poorer quality images. In fact, this was such a sensitive test that it could be used as a diagnostic indicator of the quality of the 1-dodecylamine-modified tips before proceeding to other samples.

In conclusion, this example describes an extremely useful method for making Si₃N₄ AFM tips hydrophobic. This modification procedure lowers the capillary force and improves the performance of the AFM in air. Significantly, it does not adversely affect the shape of the AFM tip and allows one to obtain lattice resolved images of hydrophilic substrates, including soft materials such as SAMs and even free-standing water, on a solid support.

### Example 4

### Multicomponent DIP PEN™ Nanolithographic Printing

This example describes the generation of multicomponent nanostructures by DIP PEN™ nanolithographic printing, and shows that patterns of two different soft materials can be generated by this technique with near-perfect alignment and 10 nm spatial resolution in an arbitrary manner. These results should open many avenues to those interested in molecule-based electronics to generate, align, and interface soft structures with each other and conventional macroscopically addressable microelectronic circuitry.

Unless otherwise specified, DIP PEN™ nanolithographic printing was performed on atomically flat Au(111) substrates using a conventional instrument (Park Scientific CP AFM) and cantilevers (Park Scientific Microlever A). The atomically flat Au(111) substrates were prepared by first heating a piece of mica at 120° C. in vacuum for 12 hours to remove possible water and then thermally evaporating 30 nm of gold onto the mica surface at 220° C. in vacuum. Using atomically flat Au(111) substrates, lines 15 nm in width can be deposited. To prevent piezo tube drift problems, a 100 μm scanner with closed loop scan control (Park Scientific) was used for all experiments. The patterning compound was coated on the tips as described in Example 1 (dipping in a solution) or by vapor deposition (for liquids and low-melting-point solids). Vapor deposition was performed by suspending the silicon nitride cantilever in a 100 ml reaction vessel 1 cm above the patterning compound (ODT). The system was closed, heated at 60° C. for 20 min, and then allowed to cool to room temperature prior to use of the coated tips. SEM analysis of tips before and after coating by dipping in a solution or by vapor deposition showed that the patterning compound uniformly coated the tips. The uniform coating on the tips allows one to deposit the patterning compound on a substrate in a controlled fashion, as well as to obtain high quality images.

Since DIP PEN™ nanolithographic printing allows one to image nanostructures with the same tool used to form them, there was the tantalizing prospect of generating nanostructures made of different soft materials with excellent registry. The basic idea for generating multiple patterns in registry by DIP PEN™ nanolithographic printing is related to analogous strategies for generating multicomponent structures by e-beam lithography that rely on alignment marks. However, the DIP PEN™ nanolithographic printing method has two distinct advantages, in that it does not make use of resists or optical methods for locating alignment marks. For example, using DIP PEN™ nanolithographic printing, one can generate 15 nm diameter self-assembled monolayer (SAM) dots of 1,16-mercaptohexadecanoic acid (MHA) on a Au(111) faceted substrate (preparation same as described above for atomically flat Au(111) substrates) by holding an MHA-coated tip in contact (0.1 nN) with the Au(111) surface for ten seconds. By increasing the scan size, the patterned dots are then imaged with the same tip by lateral force microscopy (LFM). Since the SAM and bare gold have very different wetting properties, LFM provides excellent contrast. Wilbur et al., Langmuir 11,825 (1995). Based upon the position of the first pattern, the coordinates of additional patterns can be determined, allowing for precise placement of a second pattern of MHA dots. Note the uniformity of the dots and that the maximum misalignment of the first pattern with respect to the second pattern is less than 10 nm. The elapsed time between generating the data was 10 minutes, demonstrating that DIP PEN™ nanolithographic printing, with proper control over environment, can be used to pattern organic monolayers with a spatial and pattern alignment resolution better than 10 nm under ambient conditions.

This method for patterning with multiple patterning compounds required an additional modification of the experiment described above. Since the MHA SAM dot patterns were imaged with a tip coated with a patterning compound, it is likely that a small amount of undetectable patterning compound was deposited while imaging. This could significantly affect some applications of DIP PEN™ nanolithographic printing, especially those dealing with electronic measurements on molecule-based structures. To overcome this problem, micron-scale alignment marks drawn with an MHA-coated tip were used to precisely place nanostructures in a pristine area on the Au substrate. In a typical experiment, an initial pattern of 50 nm parallel lines comprised of MHA and separated by 190 nm was prepared. This pattern was 2 μm away from the exterior alignment marks. Note that an image of these lines was not taken to avoid contamination of the patterned area. The MHA-coated tip was then replaced with an ODT-coated tip. This tip was used to locate the alignment marks, and then precalculated coordinates based upon the position of the alignment marks were used to pattern the substrate with a second set of 50 nm parallel ODT SAM lines. Note that these lines were placed in interdigitated fashion and with near-perfect registry with respect to the first set of MHA SAM lines.

There is one unique capability of DIP PEN™ nanolithographic printing referred to as “overwriting.” Overwriting involves generating one soft structure out of one type of patterning compound and then filling in with a second type of patterning compound by raster scanning across the original nanostructure. As a further proof-of concept experiment aimed at demonstrating the multiple-patterning-compound, high-registry, and overwriting capabilities of DIP PEN™ nanolithographic printing over moderately large areas, a MHA-coated tip was used to generate three geometric structures (a triangle, a square, and a pentagon) with 100 nm line widths. The tip was then changed to an ODT-coated tip, and a 10 μm by 8.5 μm area that comprised the original nanostructures was overwritten with the ODT-coated tip by raster scanning 20 times across the substrate (contact force˜0.1 nN). Since water was used as the transport medium in these experiments, and the water solubilities of the patterning compounds used in these experiments are very low, there was essentially no detectable exchange between the molecules used to generate the nanostructure and the ones used to overwrite on the exposed gold.

In summary, the high-resolution, multiple-patterning-compound registration capabilities of DIP PEN™ nanolithographic printing have been demonstrated. On an-atomically flat Au(111) surface, 15 nm patterns were generated with a spatial resolution better than 10 nm. Even on a rough surface such as amorphous gold, the spatial resolution was better than conventional photolithographic and e-beam lithographic methods for patterning soft materials.

### Example 5

### Use Of DIP PEN™ Nanolithographic Printing To Generate Resists

The suitability of DIP PEN™ nanolithographic™ printing-generated nanostructures as resists for generating three-dimensional multilayered solid-state structures by standard wet etching techniques was evaluated in a systematic study, the results of which are reported in this example. In this study, was used to deposit alkylthiol monolayer resists on Au/Ti/Si substrates. Subsequent wet chemical etching yielded the targeted three-dimensional structures. Many spatially separated patterns of the monolayer resists can be deposited by DIP PEN™ nanolithograph™ printing on a single AU/Ti/Si chip and, thus, the effects of etching conditions can be examined on multiple features in combinatorial fashion.

In a typical experiment in this study, DIP PEN™ nanolithographic™ printing was used to deposit alkylthiols onto an Au/Ti/Si substrate. It has been well established that alkyithiols form well-ordered mono layers on Au thin films that protect the underlying Au from dissolution during certain wet chemical etching procedures (Xia et al., Chem. Mater., 7:23 32 (1995); Kumar et al., J. Am. Chem. Soc., 114:9188 (1992)), and this appears to also hold true for DIP PEN™ nanolithographic™ printing-generated resists (see below). Thus, the Au, Ti, and SiO₂ which were not protected by the monolayer could be removed by chemical etchants in a staged procedure. This procedure yielded “first-stage” three-dimensional features: multilayer, Au-topped features on the Si substrate. Furthermore, “second-stage” features were prepared by using the remaining Au as an etching resist to allow for selective etching of the exposed Si substrate. Finally, the residual Au was removed to yield final-stage all-Si features. Thus, DIP PEN™ nanolithographic printing can be combined with wet chemical etching to yield three-dimensional features on Si(100) wafers with at least one dimension on the sub-100 nm length scale.

The procedure used to prepare nanoscale features on Si wafers can be diagramed. First, polished single-crystalline Si(100) wafers were coated with 5 nm of Ti, followed by 10 nm of Au by thermal evaporation. The Si(100) wafers (4″ diameter (1-0-0) wafers; 3-4.9 ohm/cm resistivity; 500-550 μm thickness) were purchased from Silicon Quest International, Inc. (Santa Clara, Calif.). Thermal evaporation of 5 nm of Ti (99.99%; Alfa Aesar; Ward Hill, Mass.) followed by 10 nm of Au (99.99%; D. F. Goldsmith; Evanston, Ill.) was accomplished using an Edwards Auto 306 Turbo Evaporator equipped with a turbopump (Model EXT510) and an Edwards FTM6 quartz crystal microbalance to determine film thickness. Au and Ti depositions were conducted at room temperature at a rate of 1 nm/second and a base pressure of <9×10⁻⁷ mb.

After Au evaporation, the following procedure was performed on the substrates: a) DIP PEN™ nanolithographic printing was used to deposit patterns of ODT, b) Au and Ti were etched from the regions not protected by the ODT monolayers using a previously reported ferri/ferrocyanide based etchant (Xia et al., Chem. Mater., 7:2332 (1995)), c) residual Ti and SiO₂ were removed by immersing the sample into a 1% HF solution (note: this procedure also passivates the exposed Si surfaces with respect to native oxide growth) (Ohmi, J. Electrochem. Soc., 143:2957(1996)), and d) the remaining Si was etched anisotropically by minor modifications of a previously reported basic etchant (Seidel et al., J. Electrochem. Soc., 137:3612 (1990)). The topography of the resulting wafers was evaluated by AFM and SEM.

All DIP PEN™ nanolithographic printing and all AFM imaging experiments were carried out with a Thermomicroscopes CP AFM and conventional cantilevers (Thermomicroscopes sharpened Microlever A, force constant 0.05 N/m, Si₃N₄). A contact force of 0.5 nN was typically used for DIP PEN™ nanolithographic printing patterning. To minimize piezo tube drift problems, a 100-μm scanner with closed loop scan control was used for all of the experiments. For DIP PEN™ nanolithographic printing, the tips were treated with ODT in the following fashion: 1) tips were soaked in 30% H₂O,:H,SO₄ (3:7) (caution: this mixture reacts violently with organic material) for 30 minutes, 2) tips were rinsed with water, 3) tips were heated in an enclosed canister (approximately 15 cm³ internal volume) with 200 mg ODT at 60° C. for 30 minutes, and 4) tips were blown dry with compressed difluoroethane prior to use. Typical ambient imaging conditions were 30% humidity and 23° C., unless reported otherwise. Scanning electron microscopy (SEM) was performed using a Hitachi SEM equipped with EDS detector.

A standard ferri/ferrocyanide etchant was prepared as previously reported (Xia et al., Chem. Mater., 7:2332 (1995)) with minor modification: 0.1 MNa₁S,O₃, 1.0 M KOH, 0.01 M K₃Fe(CN)₅, 0.001 M K₄Fe(CN)₆ in nanopure water. Au etching was accomplished by immersing the wafer in this solution for 2-5 minutes while stirring. The HF etchant (1% (v:v) solution in nanopure water) was prepared from 49% HF and substrates were agitated in this solution for 10 seconds. Silicon etching was accomplished by immersing the wafer in 4 M KOH in 15% (v:v) isopropanol in nanopure water at 55° C. for 10 seconds while stirring (Seidel et al., J. Electrochem. Soc., 137:3612 (1990)). Final passivation of the Si substrate with respect to SiO, growth was achieved by immersing the samples in 1% HF for 10 seconds with mild agitation. Substrates were rinsed with nanopure water after each etching procedure. To remove residual Au, the substrates were cleaned in O₂ plasma for 3 minutes and soaked in aqua regia (3:1 HCl:HNO₃) for 1 minute, followed by immersing the samples in 1% HF for 10 seconds with mild agitation.

Analysis shows the AFM topography images of an AU/Ti/Si chip patterned according to the procedure outlined above. This image shows four pillars with a height of 55 nm formed by etching an Au/Ti/Si chip patterned with four equal-sized dots of ODT with center-to-center distances of 0.8 μm. Each ODT dot was deposited by holding the AFM tip in contact with the Au surface for 2 seconds. Although the sizes of the ODT dots were not measured prior to etching, their estimated diameters were approximately 100 nm. This estimate is based upon the measured sizes of ODT “test” patterns deposited with the same tip on the same surface immediately prior to deposition of the ODT dots corresponding to the shown pillars. The average diameter of the shown pillar tops was 90 nm with average base diameter of 240 nm. Analysis shows a pillar (55 nm height, 45 nm top diameter, and 155 nm base diameter) from a similarly patterned and etched region on the same Au/Ti/Si substrate. The cross-sectional topography trace across the pillar diameter-showed a flat top and symmetric sidewalls. The shape of the structure may be convoluted by the shape of the AFM tip (approximately 10 nm radius of curvature), resulting in side widths as measured by AFM which may be larger than the actual widths.

Additionally, a Au/Ti/Si substrate was patterned with three ODT lines drawn by DIP PEN™ nanolithographic printing (0.4 μm/second, estimated width of each ODT line is 100 nm) with 1 μm center-to-center distances. Analysis shows the AFM topography image after etching this substrate. The top and base widths are 65 nm and 415 nm, respectively, and line heights are 55 nm. Analysis shows a line from a similarly patterned and etched region on the same Au/Ti/Si wafer, with a 50 nm top width, 155 nm base width, and 55 nm height. The cross-sectional topography trace across the line diameter shows a flat top and symmetric sidewalls.

Analysis shows the feature-size variation possible with this technique. The ODT-coated AFM tip was held in contact with the surface for varying lengths of time (16-0.062 seconds) to generate various sized dots with 2 μm center-to-center distances which subsequently yielded etched three-dimensional structures with top diameters ranging from 1.47 μm to 147 nm and heights of 80 nm. The top diameters as measured by SEM differed by less than 15% from the diameters measured from the AFM images. Additionally, energy dispersive spectroscopy (EDS) showed the presence of Au on the pillar tops whereas Au was not observed in the areas surrounding the elevated micro- and nanostructures. As expected, the diameters of the micro- and nano-trilayer structures correlated with the size of the DIP PEN™ nanolithographic printing-generated resist features, which was directly related to tip-substrate contact time. Line structures were also fabricated in combinatorial fashion. ODT lines were drawn at a scan rate varying from 0.2-2.8 μm/second with 1 μm center-to-center distances. After etching, these resists afforded trilayer structures, all with a height of 80 nm and top line widths ranging from 505 to 50 nm. The field emission scanning electron micrograph of the patterned area looks comparable to the AFM image of the same area with the top widths as determined by the two techniques being within 15% of one another.

In conclusion, it has been demonstrated that DIP PEN™ nanolithographic printing can be used to deposit monolayer-based resists with micron to sub-100 nm dimensions on the surfaces of Au/Ti/Si trilayer substrates. These resists can be used with wet chemical etchants to remove the unprotected substrate layers, resulting in three-dimensional solid-state feature with comparable dimensions. It is important to note that this example does not address the ultimate resolution of solid-state nano structure fabrication by means of DIP PEN™ nanolithographic printing. Indeed, it is believed that the feature size will decrease through the use of new “inks” and sharper “pens.” Finally, this work demonstrates the potential of using DIP PEN™ nanolithographic printing to replace the complicated and more expensive hard lithography techniques (e.g. e-beam lithography) for a variety of solid-state nanolithography applications.

### Example 6

### Multi-Pen Nanoplotter For Serial And Parallel DIP PEN™ Nanolithographic Printing

Herein, a method for doing parallel or single pen soft nanolithography using an array of cantilevers and a conventional AFM with a single feed back system is reported.

There is a key scientific observation that allows one to transform DIP PEN™ nanolithographic printing from a serial to parallel process without substantially complicating the instrumentation required. It has been discovered that features (e.g. dots and lines) generated from inks such as 1-octadecanethiol (ODT), under different contact forces that span a two-order of magnitude range, are virtually identical with respect to diameter and line-width, respectively. Surprisingly, even patterning experiments conducted with a small negative contact force, where the AFM tip bends down to the surface, exhibit ink transport rates that are comparable to experiments executed with the tip-substrate contact force as large as 4 nN. These experiments show that, in DIP PEN™ nanolithographic writing, the ink molecules may migrate from the tip through the meniscus to the substrate by diffusion, and the tip is directing molecular flow.

The development of an eight pen nanoplotter capable of doing parallel DIP PEN™ nanolithographic printing is described in this example. Significantly, since DIP PEN™ nanolithographic printing line width and writing speed are independent of contact force, this has been accomplished in a configuration that uses a single tip feedback system to monitor a tip with dual imaging and writing capabilities (designated the “imaging tip”). In parallel writing mode, all other tips reproduce what occurs at the imaging tip in passive fashion. Experiments that demonstrate eight-pen parallel writing, ink and rinsing wells, and “molecular corralling” by means of a nanoplotter-generated structure are reported.

All experiments were performed on a Thermomicroscopes MS AFM equipped with a closed loop scanner that minimizes thermal drift. Custom DIP PEN™ nanolithographic printing software (described above) was used to drive the instrument. The instrument has a 200 mm ×200 mm sample holder and an automated translation stage.

The intention in transforming DIP PEN™ nanolithographic printing into a parallel process was to create an SPL method that allows one to generate multiple single-ink patterns in parallel or a single multiple-ink pattern in series. This tool would be the nanotechnologist's equivalent of a multiple-pen nanoplotter with parallel writing capabilities. To accomplish this goal, several modifications of the AFM and DIP PEN™ nanolithographic printing process were required.

First, a tilt stage (purchased from Newport Corporation) was mounted on the translation stage of the AFM. The substrate to be patterned was placed in the sample holder, which was mounted on the tilt stage. This arrangement allows one to control the orientation of the substrate with respect to the ink coated tips which, in turn, allows one to selectively engage single or multiple tips during a patterning experiment.

Second, ink wells, which allow one to individually address and ink the pens in the nanoplotter, were fabricated. Specifically, it has been found that rectangular pieces of filter paper soaked with different inks or solvents can be used as ink wells and rinsing wells, respectively. The filter-paper ink and rinsing wells were located on the translation stage proximate the substrate. An AFM tip can be coated with a molecular ink of interest or rinsed with a solvent simply by making contact with the appropriate filter-paper ink or rinsing well for 30 seconds (contact force=1 nN).

Finally, a multiple tip array was fabricated simply by physically separating an array of cantilevers from a commercially available wafer block containing 250 individual cantilevers (Thermomicroscopes Sharpened Microlevers C, force constant=0.01 nN), and then, using that array as a single cantilever. The array was affixed to a ceramic tip carrier that comes with the commercially acquired mounted cantilevers and was mounted onto the AFM tip holder with epoxy glue.

For the sake of simplicity, experiments involving only two cantilevers in the array will be described first. In parallel writing, one tip, designated “the imaging tip,” is used for both imaging and writing, while the second tip is used simply for writing. The imaging tip is used the way a normal AFM tip is used and is interfaced with force sensors providing feedback; the writing tips do not need feedback systems. In a patterning experiment, the imaging tip is used to determine overall surface topology, locate alignment marks generated by DIP PEN™ nanolithographic printing, and lithographically pattern molecules in an area with coordinates defined with respect to the alignment marks (Example 4 and Hong et al., Science, 286:523 (1999)). With this strategy, the writing tip(s) reproduce the structure generated with the imaging tip at a distance determined by the spacing of the tips in the cantilever array (600 μm in the case of a two pen experiment).

In a typical parallel, multiple-pen experiment involving a cantilever array, each tip was coated with an ink by dipping it into the appropriate ink well. This was accomplished by moving the translation stage to position the desired ink well below the tip to be coated and lowering the tip until it touched the filter paper. Contact was maintained for 30 seconds, contact force=1 nN. To begin parallel patterning, the tilt stage was adjusted so that the writing tip was 0.4 μm closer to the sample than the imaging tip. The tip-to-sample distances in an array experiment can be monitored with the Z-stepper motor counter. The laser was placed on the imaging tip so that during patterning both tips were in contact with the surface.

The first demonstration of parallel writing involved two tips coated with the same ink, ODT. In this experiment, two one-molecule-thick nanostructures comprised of ODT were patterned onto a gold surface by moving the imaging tip along the surface in the form of a square (contact force˜0.1 nN; relative humidity 30%; writing speed=0.6 μm/sec). Note that the line-widths are nearly identical and the nanostructure registration (orientation of the first square with respect to the second) is near-perfect.

Parallel patterning can be accomplished with more than one ink. In this case the imaging tip was placed in a rinsing well to remove the ODT ink and then coated with 16-mercaptohexadecanoic acid (MBA) by immersing it in an MBA ink well. The parallel multiple-ink experiment was then carried out in a manner analogous to the parallel single ink experiment under virtually identical conditions. The two resulting nanostructures can be differentiated based upon lateral force but, again, are perfectly aligned due to the rigid, fixed nature of the two tips. Interestingly, the line-widths of the two patterns were identical. This likely is a coincidental result since feature size and line width in a DIP PEN™ nanolithographic printing experiment often depend on the transport properties of the specific inks and ink loading.

A remarkable feature of this type of nanoplotter is that, in addition to offering parallel writing capabilities, one can operate the system in serial fashion to generate customized nanostructures made of different inks. To demonstrate this capability, a cantilever array that had a tip coated with ODT and a tip coated with MHA was utilized. The laser was focused on the ODT coated tip, and the tilt stage was adjusted so that only this tip was in contact with the surface. The ODT coated tip was then used to generate the vertical sides of a cross on a Au surface (contact force˜0.1 nN; relative humidity˜30%; writing speed=1.3 μm/second). The laser was then moved to the MBA coated tip, and the tilt stage was readjusted so that only this tip was in contact with surface. The MHA tip was then used to draw the 30 nm wide horizontal sides of the nanostructure (“nano” refers to line width). Microscopic ODT alignment marks deposited on the periphery of the area to be patterned were used to locate the initial nanostructure as described above (see also Example 4 and Hong et al., Science, 286:523 (1999)).

This type of multiple ink nanostructure with a bare gold interior would be very difficult to prepare by stamping methodologies or conventional nanolithography methods, but was prepared in five minutes with the multiple-pen nanoplotter. Moreover, this tool and these types of structures can now be used to begin evaluating important-issues involving molecular diffusion on the nanometer length scale and across nanometer wide molecule-based barriers. As a proof-of-concept, the diffusion of MHA from a tip to the surface within this type of “molecule-based corral” was examined. As a first step, a cross shape was generated with a single ink, ODT (contact force˜0.1 nN; relative humidity˜30%; writing speed=0.5 μm/second). Then, an MHA coated tip was held in contact with the surface for ten minutes at the center of the cross so that MHA molecules were transported onto the surface and could diffuse out from the point of contact. Importantly, even 80 nm wide ODT lines acted as a diffusion barrier, and MBA molecules were trapped inside the ODT cross pattern. When the horizontal sides of the molecular corral are comprised of MHA barriers, the MHA molecules diffuse from tip onto the surface and over the hydrophilic MHA barriers. Interestingly, in this two component nanostructure, the MBA does not go over the MHA barriers, resulting in an anisotropic pattern. Although it is not known yet if the corral is changing the shape of the meniscus, which in turn controls ink diffusion, or alternatively, the ink is deposited and then migrates from the point of contact to generate this structure, this type of proof-of-concept experiment shows how one can begin to discover and study important interfacial processes using this new nanotechnology tool.

The parallel nanoplotting strategy reported herein is not limited to two tips. Indeed, it has been shown that a cantilever array consisting of eight tips can be used to generate nanostructures in parallel fashion. In this case, each of the eight tips was coated with ODT. The outermost tip was designated as the imaging tip and the feedback laser was focused on it during the writing experiment. To demonstrate this concept, four separate nanostructures, a 180 nm dot (contact force˜0.1 nN, relative humidity 32 26%, contact time =1 second), a 40 nm wide line, a square and an octagon (contact force˜0.1 nN, relative humidity=26%, writing speed=0.5 μm/second) were generated and reproduced in parallel fashion with the seven passively following tips. Note that there is a less than 10% standard deviation in line width for the original nanostructures and the seven copies.

In summary, DIP PEN™ nanolithographic printing has been transformed from a serial to a parallel process and, through such work, the concept of a multiple-pen nanoplotter with both serial and parallel writing capabilities has been demonstrated. It is important to note that the number of pens that can be used in a parallel DIP PEN™ nanolithographic printing experiment to passively reproduce nanostructures is not limited to eight. Indeed, there is no reason why the number of pens cannot be increased to hundreds or even a thousand pens without the need for additional feedback systems. Finally, this work will allow researchers in the biological, chemical, physics, and engineering communities to begin using DIP PEN™ nanolithographic printing and conventional AFM instrumentation to do automated, large scale, moderately fast, high-resolution and alignment patterning of nanostructures for both fundamental science and technological applications.

### Example 7

### Use of DIP PEN™ Nanolithographic Printing to Prepare Combinatorial Arrays

In this example, the general method is to form a pattern on a substrate composed of an array of dots of an ink which will attract and bind a specific type of particle. For the present studies, MHA was used to make templates on a gold substrate, and positively-charged protonated amine- or amidine-modified polystyrene spheres were used as particle building blocks.

Gold coated substrates were prepared as described in Example 5. For in situ imaging experiments requiring transparent substrates, glass coverslips (Corning No. 1 thickness, VWR, Chicago, Ill.) were cleaned with Ar/O—, plasma for 1 minute, then coated with 2 nm of Ti and 15 nm of Au. The unpattemed regions of the gold substrate were passivated by immersing the substrate in a 1 mM ethanolic solution of another alkanethiol, such as ODT or cystamine. Minimal, if any, exchange took place between the immobilized MHA molecules and the ODT or cystamine in solution during this treatment, as evidenced by lateral force microscopy of the substrate before and after treatment with ODT.

The gold substrates were patterned with MHA to form arrays of dots. DIP PEN™ nanolithographic printing patterning was carried out under ambient laboratory conditions (30% humidity, 23° C.) as described in Example 5. It is important to note that the carboxylic acid groups in the MHA patterns were deprotonated providing an electrostatic driving force for particle assembly. (Vezenov et al., J. Am. Chem. Soc. 119:2006-2015 (1997))

Suspensions of charged polystyrene latex particles in water were purchased from either Bangs Laboratories (0.93 μm, Fishers, Ind.) or IDC Latex (1.0 μm and 190 nm, Portland, Oreg.). Particles were rinsed free of surfactant by centrifugation and redispersion twice in distilled deionized water (18.1 MΩ) purified with a Barnstead (Dubuque, Iowa) NANOpure water system. Particle assembly on the substrate was accomplished by placing a 20 μ1 droplet of dispersed particles (10% wt/vol in deionized water) on the horizontal substrate in a humidity chamber (100% relative humidity). Gentle rinsing with deionized water completed the process.

Optical microscopy was performed using the Park Scientific CP AFM optics (Thermomicroscopes, Sunnyvale, Calif.) or, for in situ imaging, an inverted optical microscope (Axiovert 100A, Carl Zeiss, Jena, Germany) operated in differential interference contrast mode (DIC). Images were captured with a Penguin 600 CL digital camera (Pixera, Los Gatos, Calif.). Intermittent-contact imaging of particles was performed with a Thermomicroscopes MS AFM using silicon ultralevers (Thermomicroscopes, spring constant=3.2 N/m). Lateral force imaging was carried out under ambient laboratory conditions (30% humidity, 23° C.) and as previously reported (Weinberger et al., Adv. Mater. 12:1600-1603 (2000)).

In a typical experiment involving 0.93 μm diameter particles, multiple templates were monitored simultaneously for particle assembly by optical microscopy. In these experiments, the template dot diameter was varied to search for optimal conditions for particle-template recognition. After 1 hour of particle assembly, the substrates were rinsed with deionized water, dried under ambient laboratory conditions, and then imaged by optical microscopy. The combinatorial experiment revealed that the optimum size of the template pad with which to immobilize a single particle of this type in high registry with the pattern was approximately 500-750 nm. It is important to note that drying of the substrate tended to displace the particles from their preferred positions on the template, an effect that has been noted by others with larger scale experiments (Aizenberg et al., Phys. Rev. Lett. 84:2997-3000 (2000)). Indeed, evidence for better, in fact near-perfect, particle organization is obtained by in situ imaging of the surface after 1 μm amine-modified particles have reacted with the template for 1 hour.

Single particle spatial organization of particles on the micron length-scale has been achieved by physical means, for instance using optical tweezers (Mio et al., Langmuir 15:8565-8568 (1999)) or by sedimentation onto e-beam lithographically patterned polymer films (van Blaaderen et al., Nature 385:321-323 (1997)). However, the DIP PEN™ nanolithographic™ printing-based method described here offers an advantage over previous methods because it provides flexibility of length scale and pattern type, as well as a means to achieve more robust particle array structures. For instance, DIP PEN™ nanolithographic printing has been used to construct chemical templates which can be utilized to prepare square arrays of 190 nm diameter amidine-modified polystyrene particles. Screening of the dried particle arrays using non-contact AFM or SEM imaging revealed that 300 nm template dots of MHA, spaced 570 nm apart, with a surrounding repulsive monolayer of cystamine, were suitable for immobilizing single particles at each site in the array. However, MHA dots of diameter and spacing of 700 nm and 850 nm resulted in immobilization of multiple particles at some sites.

Similar particle assembly experiments conducted at pH <5 or >9 resulted in random, non-selective particle adsorption, presumably due to protonation of the surface acid groups or deprotonation of particle amine or amidine groups. These experiments strongly suggested that the particle assembly process was induced by electrostatic interactions between charged particles and patterned regions of the substrate.

In conclusion, it has been demonstrated that DIP PEN™ nanolithographic printing can be used as a tool for generating combinatorial chemical templates with which to position single particles in two-dimensional arrays. The specific example of charged alkanethiols and latex particles described here will provide a general approach for creating two-dimensional templates for positioning subsequent particle layers in predefined crystalline structures that may be composed of single or multiple particle sizes and compositions. In a more general sense, the combinatorial DIP PEN™ nanolithographic™ printing method will allow researchers to efficiently and quickly form patterned substrates with which to study particle-particle and particle-substrate interactions, whether the particles are the dielectric spheres which comprise certain photonic band-gap materials, metal, semiconductor particles with potential catalytic or electronic properties, or even living biological cells and macrobiomolecules.

### Example 8

### Nanoscopic Lysozyme and Immunoglobulin Peptide and Protein Nanoarrays Generated by DIP PEN™ Nanolithographic Printing

Herein, it is described how the high resolution patterning method, DIP PEN™ nanolithographic printing, can be used to construct nanoarrays of proteins with 100 nm features. Moreover, it is demonstrated that these arrays can be fabricated with almost no detectable nonspecific binding of proteins to passivated portions of the array and that reactions involving the protein features and antigens in solution can be screened by AFM.

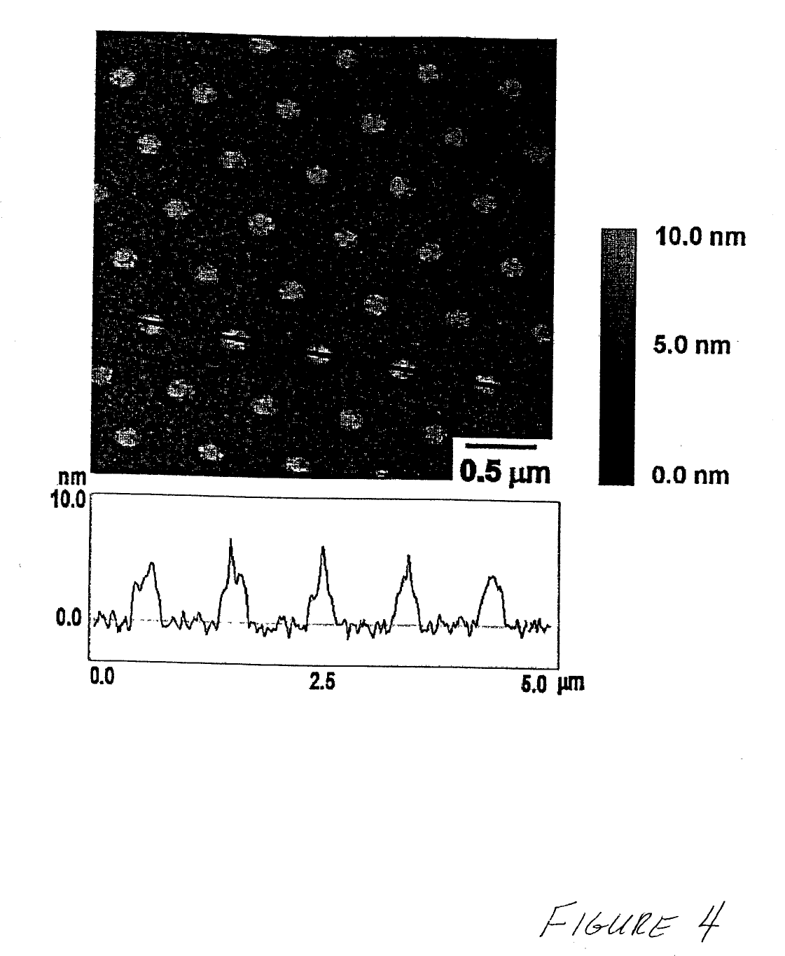

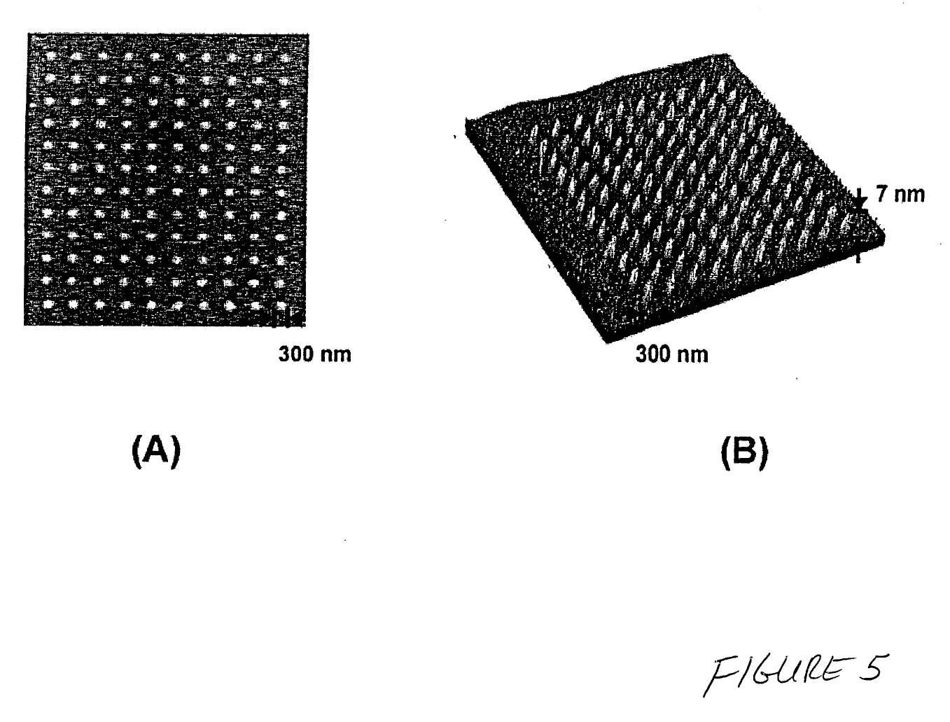

A typical protein array was fabricated by initially patterning 16-mercaptohexadecanoic acid (MHA) on a gold thin film substrate in the form of dots or grids. The features studied thus far, both lines and dots, have been as large as 350 nm (line width and dot diameter, respectively) and as small as 100 nm, FIG. 2. The areas surrounding these features were passivated with 11-mercaptoundecyl-tri(ethylene glycol) by placing a droplet of a 10 mM ethanolic solution of the surfactant on the patterned area for 45 minutes followed by copious rinsing with ethanol and, then, nanopure water. Either lysozyme or rabbit immunoglobulin G proteins were assembled on the preformed MHA patterns (FIG. 1). This was accomplished by immersing the gold substrate with an array of MHA features in a solution containing the desired protein (10 μg/mL) for 1 h. After incubation with the protein of interest, the substrate was removed and rinsed with 10 mM Tris buffer (Tris-(hydroxymethly)aminomethane), Tween-20 solution (0.05%) and, then, nanopure water.

All DIP PEN™ nanolithographic printing patterning and contact mode imaging experiments were done with a ThermoMicroscopes CP AFM interfaced with customized software and conventional Si₃N₄ cantilevers (Thermo Microscopes sharpened Microlever A, force constant=0.05 N/m). Tapping mode images were taken with a Nanoscope IIIa and MultiMode microscope from Digital Instruments. Unless otherwise mentioned, all DIP PEN™ nanolithographic printing patterning experiments were conducted under ambient conditions at 40% relative humidity and 24° C. with a tip-substrate contact force of 0.5 nN. A 100 μm scanner, with closed -loop scan control, was used for all DIP PEN™ nanolithographic printing experiments to minimize piezo tube drift and alignment problems.

Lysozyme was shown to cleanly assemble on the MHA nanopattern arrays, as evidenced by contact and tapping mode AFM, FIGS. 2B-D, respectively. Note that there is almost no evidence of nonspecific protein adsorption on the array and that height profiles suggest that between one and two layers of protein adsorb at each MHA site. Because lysozyme has an ellipsoidal shape (4.5×3.0×3.0 nm³), (see Blake et al., Nature 206, 757, 1965), it can adopt at two significantly different configurations (lying on its long axis or standing upright) on the substrate surface which can be differentiated based upon differences in height, FIG. 2C (inset). Indeed, both orientations are observed in the height profiles of the AFM experiment, as evidenced by features with either 4.5 or 3.0 nm heights. Finally, the protein can be assembled in almost any sort of array configuration, including lines and grids, FIG. 2D.

Immunoglobulin G, which has substantially different dimensions (Y-shape, height=14.5 nm, width=8.5 nm, thickness=4.0 nm³), (see Silverton et al., Proc. Natl. Acad. Sci. U.S.A. 74, 5140, 1977), exhibits qualitatively similar adsorption characteristics, FIG. 3. The basic structure of monomeric IgG is composed of two identical halves; each half has a heavy chain and a light chain. The height profile of an IgG nanoarray shows that each IgG feature is 8.0±0.7 nm (n=10) high, which is consistent with a single monolayer of the protein adsorbed onto the MHA features and is comparable to what others have seen for macroscopic features (see Browning-Kelley et al., Langmuir 13, 343, 1997; Waud-Mesthrige et al., Langmuir 15, 8580, 1999; Waud-Mesthrige et al., Biophys. J. 80 1891, 2001; Kenseth et al., Langmuir 17, 4105, 2001). Although it is not fully understood, this height also possibly can be consistent with two layers of the protein laying flat on top of each other, although this is unlikely based upon the reactivity of the arrays.

The claimed invention is not restricted to the aforementioned disclosure and working examples.

What is claimed is:

1. A protein nanoarray comprising:
a) a nanoarray substrate,
b) a plurality of dots on the substrate, the dots comprising at least one patterning compound on the substrate, and at least one protein on the patterning compound.

2. The protein nanoarray of claim 1, wherein the patterning compound is placed on the substrate by dip pen nanolithographic printing.

3. The protein nanoarray of claim 1, wherein the plurality of dots is a lattice of dots.

4. The protein nanoarray of claim 1, wherein the plurality of dots comprises at least 10 dots.

5. The protein nanoarray of claim 1, wherein the plurality of dots comprises at least 100 dots.

6. The protein nanoarray of claim 1, wherein the substrate is an insulator.

7. The protein nanoarray of claim 1, wherein the substrate is glass.

8. The protein nanoarray of claim 1, wherein the substrate is a metal, a semiconductor, a magnetic material, a polymer material, a polymer-coated substrate, or a superconductor material.

9. The protein nanoarray of claim 1, wherein the substrate is a metal.

10. The protein nanoarray of claim 1, wherein the patterning compound is chemisorbed to or covalently bound to the substrate.

11. The protein nanoarray of claim 1, wherein the patterning compound is a sulfur-containing patterning compound.

12. The protein nanoarray of claim 1, wherein the patterning compound is a sulfur-containing compound having a sulfur group at one end and a terminal reactive group at the other end.

13. The protein nanoarray of claim 1, wherein the protein is a globular protein.

14. The protein nanoarray of claim 1, wherein the protein is a fibrous protein.

15. The protein nanoarray of claim 1, wherein the protein is an enzyme.

16. The protein nanoarray of claim 1, wherein the protein is an antibody.

17. The protein nanoarray of claim 1, wherein the protein is lysozyme.

18. The protein nanoarray of claim 1, wherein the protein is an immunoglobulin.

19. The protein nanoarray of claim 1, wherein the dots have diameters of about 300 nm or less.

20. The protein nanoarray of claim 1, wherein the dots have diameters of about 100 nm or less.

21. The protein nanoarray of claim 1, wherein the patterning compound is placed on the substrate by dip pen nanolithographic printing, wherein the protein is place on the patterning compound by adsorption, wherein the substrate is a metal or insulator, wherein the protein is a globular or fibrous protein, and wherein the dots have diameters of about 1,000 nm or less.

22. The protein nanoarrray of claim 1, wherein the substrate is a metal or glass, wherein the protein is an enzyme or an antibody, and wherein the dots have diameters of about 500 nm or less.

23. The protein nanoarrray of claim 1, wherein the substrate is metal, wherein the patterning compound is a sulfur compound, wherein the protein is an enzyme or an antibody, and wherein the dots have diameters of about 300 nm or less.

24. The protein nanoarrray of claim 1, wherein the plurality of dots forms a lattice, wherein the substrate is gold, wherein the patterning compound is an alkanethiol compound, wherein the protein is an enzyme or an antibody, wherein the dots have diameters of about 100 nm or less, and wherein the substrate comprises a protein passivation compound on the substrate surrounding the dots.

25. A protein nanoarray comprising:
a) a nanoarray substrate,
b) a plurality of lines on the substrate, the lines comprising at least one patterning compound on the substrate, and at least one protein on the patterning compound.

26. The protein nanoarray of claim 25, wherein the patterning compound is placed on the substrate by dip pen nanolithographic printing.

27. The protein nanoarray of claim 25, wherein the plurality of lines is a grid of perpendicular or parallel lines.

28. The protein nanoarray of claim 25, wherein the plurality of lines comprises at least 10 lines.

29. The protein nanoarray of claim 25, wherein the plurality of lines comprises at least 100 lines.

30. The protein nanoarray of claim 25, wherein the substrate is an insulator.

31. The protein nanoarray of claim 25, wherein the substrate is glass.

32. The protein nanoarray of claim 25, wherein the substrate is a metal.

33. The protein nanoarray of claim 25, wherein the patterning compound is chemisorbed to or covalently bound to the substrate.

34. The protein nanoarray of claim 25, wherein the patterning compound is a sulfur compound.

35. The protein nanoarray of claim 25, wherein the patterning compound is a sulfur compound having a thiol group at one end and a terminal reactive group at the other end.

36. The protein nanoarray of claim 25, wherein the protein is a globular protein.

37. The protein nanoarray of claim 25, wherein the protein is a fibrous protein.

38. The protein nanoarray of claim 25, wherein the protein is an enzyme.

39. The protein nanoarray of claim 25, wherein the protein is an antibody.

40. The protein nanoarray of claim 25, wherein the protein is lysozyme.

41. The protein nanoarray of claim 25, wherein the protein is an immunoglobulin.

42. The protein nanoarray of claim 25, wherein the lines have widths of about 300 nm or less.

43. The protein nanoarray of claim 25, wherein the lines have widths of about 100 nm or less.

44. The protein nanoarray of claim 25, wherein the patterning compound is deposited on the substrate by dip pen nanolithographic printing, wherein the protein is adsorbed to the patterning compound, wherein the substrate is a an insulator or metal, wherein the protein is a globular or fibrous protein, and wherein the lines have widths of about 1,000 nm or less.

45. The protein nanoarray of claim 25, wherein the patterning compound is deposited on the substrate by dip pen nanolithographic printing, wherein the protein is adsorbed to the patterning compound, wherein the substrate is a an insulator or metal, wherein the protein is a globular or fibrous protein, wherein the patterning compound is a sulfur compound, and wherein the lines have widths of about 500 nm or less.

46. The protein nanoarray of claim 25, wherein the substrate is a an insulator or metal, wherein the protein is a globular or fibrous protein, wherein the patterning compound is a sulfur compound, wherein the lines have widths of about 500 nm or less, and wherein the substrate comprises a protein passivation compound on the substrate between the lines.

47. The protein nanoarrray of claim 25, wherein the substrate is a metal, wherein the protein is an enzyme or an antibody, and wherein the lines have widths of about 500 nm or less.

48. The protein nanoarrray of claim 25, wherein the substrate is gold, wherein the lines comprise a thiol compound on the substrate, wherein the protein is an enzyme or an antibody, and wherein the lines have widths of about 300 nm or less.

49. The protein nanoarrray of claim 25, wherein the substrate is a metal or insulator, wherein the patterning compound is deposited onto the substrate by dip pen nanolithographic printing followed by passivation of the substrate, wherein the protein is an enzyme or an antibody, and wherein the lines have widths of about 100 nm or less.

50. A protein nanoarray comprising:
a) a nanoarray substrate,
b) a plurality of patterns on the substrate, the patterns comprising at least one patterning compound on the substrate and at least one protein adsorbed to each of the patterns.

51. A protein nanoarray according to claim 50, wherein the patterns are formed by dip pen nanolithographic printing.

52. A protein nanoarray according to claim 50, wherein the patterns are formed by dip pen nanolithographic printing on the substrate, followed by passivation of the substrate, followed by adsorption of the protein to the patterning compound.

53. A protein nanoarray according to claim 50, wherein the patterns comprise at least one patterning compound which is chemisorbed to or covalently bound to the substrate.

54. The protein nanoarray according to claim 50, wherein the patterns are dots having diameters of about 500 nm or less.

55. The protein nanoarray according to claim 50, wherein the patterns are dots having diameters of about 300 nm or less.

56. The protein nanoarray according to claim 50, wherein the patterns are dots having diameters of about 100 nm or less.

57. The protein nanoarray according to claim 50, wherein the patterns are lines having widths of about 500 nm or less.

58. The protein nanoarray according to claim 50, wherein the patterns are lines having widths of about 300 nm or less.

59. The protein nanoarray according to claim 50, wherein the patterns are lines having widths of about 100 nm or less.

60. A peptide nanoarray comprising:
a) a nanoarray substrate,
b) a plurality of dots on the substrate, the dots comprising at least one compound on the substrate, and at least one peptide adsorbed to each of the dots.

61. The peptide nanoarray of claim 60, wherein the plurality of dots is a lattice of dots.

62. A peptide nanoarray according to claim 60, wherein the peptide is an oligopeptide.

63. A peptide nanoarray according to claim 60, wherein the peptide is a polypeptide.

64. A peptide nanoarray according to claim 60, wherein the peptide is a compound comprising at least three peptide bonds.

65. A peptide nanoarray according to claim 60, wherein the peptide is a compound comprising ten or less peptide bonds.

66. A peptide nanoarray according to claim 60, wherein the peptide is a compound comprising at least one hundred peptide bonds.

67. A peptide nanoarray according to claim 60, wherein the peptide is a compound comprising about one hundred to about 300 peptide bonds.

68. A peptide nanoarray according to claim 60, wherein the peptide is a compound comprising at least five hundred peptide bonds.

69. A peptide nanoarray according to claim 60, wherein the compound is put on the substrate by dip pen nanolithographic printing, and the compound is chemisorbed to or covalently bonded to the substrate.

70. A peptide nanoarray comprising:
a) a nanoarray substrate,
b) a plurality of lines on the substrate, the lines comprising at least one compound on the substrate and at least one peptide on the compound.

71. A peptide nanoarray according to claim 70, wherein the peptide is an oligopeptide or a polypeptide.

72. A peptide nanoarray according to claim 70, wherein the peptide is a polypeptide.

73. A peptide nanoarray according to claim 70, wherein the peptide is a compound comprising at least three peptide bonds.

74. A peptide nanoarray according to claim 70, wherein the peptide is a compound comprising ten or less peptide bonds.

75. A peptide nanoarray according to claim 70, wherein the peptide is a compound comprising more than ten peptide bonds.

76. A peptide nanoarray according to claim 70, wherein the peptide is a compound comprising at least one hundred peptide bonds.

77. A peptide nanoarray according to claim 70, wherein the peptide is a compound comprising about one hundred to about 300 peptide bonds.

78. A peptide nanoarray according to claim 70, wherein the peptide is a compound comprising at least five hundred peptide bonds.

79. A peptide nanoarray according to claim 70, wherein the compound is placed on the substrate by dip pen nanolithographic printing, and the compound is chemisorbed or covalently bound to the substrate.

80. A peptide nanoarray comprising:
a nanoarray substrate,
at least one pattern on the substrate, the pattern comprising a patterning compound covalently bound to or chemisorbed to the substrate, the pattern comprising a peptide adsorbed on the patterning compound.

81. The peptide nanoarray according to claim 80, wherein the pattern is a dot or line.

82. The peptide nanoarray according to claim 81, wherein the nanoarray comprises at least two patterns on the substrate.

83. The peptide nanoarray according to claim 82, wherein the pattern is a dot or line, the dot having a diameter of 500 nm or less, the line having a width of 500 nm or less.

84. The peptide nanoarray according to claim 83, wherein the patterning compound is a sulfur compound.

85. The peptide nanoarray according to claim 84, wherein the patterning compound is deposited onto the substrate by dip pen nanolithographic printing.

86. The peptide nanoarray according to claim 85, wherein the peptide has at least 100 peptide bonds.

87. The peptide nanoarray according to claim 85, wherein the pattern is located on an etched pillar.

88. The peptide nanoarray of claim 80, wherein the peptide is a protein, a polypeptide, or an oligopeptide, and the pattern is in the form of a dot or line.

89. The peptide nanoarray of claim 80, wherein the peptide is a protein, a polypeptide, or an oligopeptide, the pattern is in the form of a dot or line, and the nanoarray comprises at least 10 patterns in an array or grid.

90. A method for making a nanoarray comprising:
patterning a compound on a nanoarray surface by dip pen nanolithographic printing to form a pattern; and
assembling at least one peptide onto the pattern.

91. A method according to claim 90, wherein the peptide is a protein.

92. A method according to claim 90, wherein the peptide is a polypeptide.

93. A method according to claim 90, wherein the peptide is an oligopeptide.

94. The method according to claim 91, wherein the compound after patterning on the surface is capable of adsorbing the protein.

95. The method of claim 91, wherein said compound after patterning on the surface is capable of forming a covalent bond, an ionic bond, a hydrogen bond, or an electrostatic interaction with the protein.

96. The method of claim 91, wherein said compound after patterning has a terminal functional group which binds to the protein.

97. The method of claim 90, wherein said compound is selected from the group consisting of a sulfur-containing compound, a silicon-containing compound, a carboxylic acid-containing compound, an aldehyde-containing compound, an alcohol compound, an alkoxy-containing compound, a vinyl-containing compound, an amine compound, a nitrile compound, and an isonitrile compound.

98. The method of claim 90, wherein the compound is a sulfur-containing compound.

99. The method of claim 91, wherein the protein is a globular protein.

100. The method of claim 91, wherein the protein is a fibrous protein.

101. The method of claim 91, wherein the protein is a water-soluble protein.

102. The method of claim 91, wherein the protein is a water-insoluble protein.

103. The method of claim 91, wherein the protein is an enzyme.

104. The method of claim 91, wherein the protein is an antibody.

105. The method of claim 90, wherein the patterning is carried out to form a plurality of patterns, and the patterns are lines or dots.

106. The method of claim 90, wherein the pattern is a line or dot.

107. The method of claim 106, wherein the line has a width less than about 1,000 nm and the dot has a diameter of less than about 1,000 nm.

108. The method of claim 107, wherein the line has a width less than about 350 nm and the dot has a diameter of less than about 350 nm.

109. The method of claim 107, wherein the line has a width less than about 100 nm and the dot has a diameter of less than about 100 nm.

110. The method of claim 90, further comprising passivating areas of the surface on which said compound was not patterned.

111. The method of claim 90, wherein the assembling step comprises immersing the patterned surface in a solution of peptide.

112. The method of claim 90, wherein the compound is a sulfur-containing compound, wherein the peptide is a globular or fibrous protein, and wherein the pattern is a dot or line.

113. The method according to claim 90, wherein the compound is a sulfur-containing compound, wherein the peptide is a protein, wherein the pattern is a dot or line, and wherein said surface is passivated after patterning.

114. The method according to claim 90, wherein the compound is a sulfur-containing compound, wherein the protein is a globular or fibrous protein, wherein the patterning is carried out multiple times to form a plurality of dots or lines, wherein said surface is passivated after patterning, wherein the surface is a metal or insulating surface, and wherein the diameter of each dot is less than about 1,000 nm and wherein the width of each line is less than about 1,000 nm.

115. The method of claim 114, wherein diameter and width are less than about 500 nm.

116. The method of claim 114, wherein the diameter and width are less than about 100 nm.

117. A method according to claim 90, wherein the peptide is a polypeptide and the pattern is a dot or line.

118. A method according to claim 90, wherein the peptide is a polypeptide and the pattern is a dot having a diameter of 500 nm or less, or a line having a width of 500 nm or less.

119. A method according to claim 90, wherein the peptide is a polypeptide and the pattern is a dot having a diameter of 500 nm or less, or a line having a width of 100 nm or less.

120. A method comprising:
patterning a compound on a nanoarray surface using a coated atomic force microscope tip to form a plurality of nanoscale patterns, and
adsorbing one or more peptides onto the patterns.

121. A method according to claim 120, wherein the peptides are proteins.

122. A method according to claim 120, wherein the peptides are polypeptides.

123. A method according to claim 120, wherein patterning is carried out to form a plurality of dots or lines.

124. A method according to claim 120, wherein the compound is a sulfur compound.

125. A method according to claim 120, further comprising the step of overwriting peptide on the pattern of one or more peptide.

126. A method according to claim 120, wherein patterning is carried out with multiple coated atomic microscope tips.

127. A method according to claim 126, wherein the dots have diameters and the lines have widths of 300 nm or less.

128. A method according to claim 126, wherein the patterning is carried out to make a plurality of at least ten dots or lines.

129. A method according to claim 126, further comprising passivating the surface after patterning.

130. A method for making protein nanoarrays with nanoscopic features comprising:
assembling one or more proteins onto a preformed pattern nanoarray, wherein the protein becomes adsorbed to the pattern nanoarray and the pattern nanoarray is formed by dip pen nanolithographic printing.

131. A method for making peptide arrays with nanoscopic features comprising:
assembling one or more peptides onto a preformed nanoarray pattern, wherein the peptide becomes adsorbed to the nanoarray pattern and the nanoarray pattern is formed by dip pen nanolithographic printing.

132. A method for making a nanoscale array of protein comprising:
depositing by dip-pen nanolithographic printing a patterning compound on a nanoarray surface;
passivating the undeposited regions of the surface with a passivation compound,
exposing said surface having the patterning compound and the passivation compound to a solution comprising at least one protein;
removing said surface from said solution of protein, wherein said surface comprises a nanoscale array of protein.

133. A nanoarray prepared by the method according to claim 90.

134. An array comprising a plurality of nanoscale patterns with adsorbed protein formed by the method according to claim 120.

135. A protein nanoarray prepared by the method according to claim 130.

136. A peptide array prepared by the method according to claim 131.

137. A nanoscale array of protein prepared by the method according to claim 132.

138. A submicrometer array comprising:
a plurality of discrete sample areas arranged in a pattern on a substrate,
each sample area being a predetermined shape,
at least one dimension of each of the sample areas, other than depth, being less than about one micron,
wherein each of the sample areas comprise a patterning compound on the substrate and a peptide on the patterning compound.

139. The array according to claim 138, wherein the peptide is a protein.

140. The array according to claim 138, wherein the predetermined shape is a dot or line.

141. The array according to claim 138, wherein the plurality of discrete sample areas is at least 100 sample areas.

142. The array according to claim 138, wherein the plurality of discrete sample areas is put on the substrate by methods comprising dip pen nanolithographic printing.

143. The array according to claim 138, wherein the dimension is less than 500 nm.

144. The array according to claim 138, wherein the dimension is less than about 300 nm.

145. The array according to claim 138, wherein the array is prepared by dip pen nanolithographic printing of a patterning compound onto the substrate followed by peptide adsorption onto the patterning compound.

146. A peptide nanoarray comprising:
a) a nanoarray substrate,
b) a plurality of patterns on the substrate, the patterns comprising at least one patterning compound on the substrate having a terminal functional group and at least one peptide bound to each of the patterns through the terminal functional group.

147. The peptide nanoarray according to claim 146, wherein the peptide is a protein.

148. The peptide nanoarray according to claim 146, wherein the patterns are line having widths of 500 nm or less, or dots having diameters of 500 nm or less.

149. The peptide nanoarray according to claim 146, wherein the patterning compounds are chemisorbed or covalently bonded to the substrate by dip pen nanolithographic printing.

150. A method for detecting the presence or absence of a target in a sample, comprising:
exposing a nanoarray substrate surface to a sample, the substrate surface comprising a plurality of one or more peptides assembled on one or more compounds anchored to said substrate surface,
observing whether a change in a property occurs upon the exposure which indicates the presence or absence of the target in the sample.

151. The method according to claim 150, wherein the nanoarray substrate surface is prepared with use of dip pen nanolithographic printing.

152. The method according to claim 150, wherein the change in property is a change in shape, stickiness, height, or a combination thereof.

153. The method according to claim 150, wherein the peptide is a protein.

154. The method according to claim 150, wherein the nanoarray substrate surface comprises a plurality of patterns comprising the peptide and the compound, the patterns having a length or width less than about 500 nm.

155. A method for detecting the presence or absence of a target in a sample, comprising:
exposing a nanoarray substrate surface to (i) the sample which may or may not comprise the target, and (ii) a molecule that is capable of interacting with the target, wherein the substrate surface comprises one or more peptides assembled on one or more compounds anchored to said substrate surface and the peptides are capable of binding to the target,
detecting the presence or absence of the target in the sample based on interaction of the molecule with the target, the target being bound to the peptide.

156. The method according to claim 155, wherein the nanoarray substrate surface is prepared with use of dip pen nanolithographic printing.

157. The method according to claim 155, wherein the peptide is a protein.

158. The method according to claim 155, wherein the nanoarray substrate surface comprises a plurality of patterns comprising the peptide and the compound, the patterns having a length or width less than about 500 nm.

159. The method according to claim 155, wherein the nanoarray substrate surface is prepared with use of dip pen nanolithographic printing, wherein the nanoarray substrate surface comprises a plurality of patterns comprising the peptide and the compound, the patterns having a length or width less than about 500 nm, and wherein the peptide is a protein.

160. A method for detecting the presence or absence of a target in a sample, comprising
measuring at least one dimension of one or more nanoscale deposits of peptides on a surface;
exposing said surface to said sample; and
detecting whether a change occurs in the dimension of the one or more nanoscale deposits of peptides which indicates the presence or absence of the target.

161. The method according to claim 160, wherein the nanoscale deposit of peptide is prepared with use of dip pen nanolithographic printing.

162. The method according to claim 162, wherein the peptide is a protein.

163. The method according to claim 162, wherein the nanoscale deposit of peptide comprises the peptide and a patterning compound, the deposit being a pattern having a length or width less than about 500 nm.

164. The method according to claim 163, wherein the nanoscale deposit of peptide is prepared with use of dip pen nanolithographic printing, and wherein the peptide is a protein.

165. The method according to claim 160, wherein the dimension is height.
